(19) United States
(12) Patent Application Publication
(10) Pub. No.: US 2023/0298703 A1
(43) Pub. Date: Sep. 21, 2023

(54) SINGLE-PASS METHYLATION MAPPING

(71) Applicant: Illumina Software, Inc., San Diego, CA (US)

(72) Inventors: John Cooper Roddey, San Diego, CA (US); Asaf Levy, San Diego, CA (US); Mengchi Wang, San Diego, CA (US); Adam Birnbaum, San Diego, CA (US); Brian Y. Tsui, San Diego, CA (US); Michael Ruehle, San Diego, CA (US)

(21) Appl. No.: 18/183,514

(22) Filed: Mar. 14, 2023

Related U.S. Application Data
(60) Provisional application No. 63/320,110, filed on Mar. 15, 2022.

(51) Int. Cl. G16B 30/10; C12Q 1/6827; G16B 20/10

(52) U.S. Cl. CPC G16B 30/10; C12Q 1/6827; G16B 20/10

(57) ABSTRACT

Disclosed herein include systems, machines, devices, and methods for single-pass methylation mapping. C-to-T converted sequence reads and G-to-A converted sequence reads generated from a sample subjected to a methylation assay can be mapped to a mapping reference sequence comprising a C-to-T converted reference sequence and a G-to-A converted reference sequence generated to a reference genome sequence. The counts of Cs and Ts of sequence reads mapped to each of one or more positions with Cs in the reference genome sequence can be used to determine whether the position is a methylated C or an unmethylated C in the sample.

### RELATED APPLICATIONS

This application claims the benefit under 35 U.S.C. § 119(e) of U.S. Provisional Patent Application Ser. No. 63/320,110, filed Mar. 15, 2022; the content of this related application is incorporated herein by reference in its entirety for all purposes.

### COPYRIGHT NOTICE

A portion of the disclosure of this patent document contains material which is subject to copyright protection. The copyright owner has no objection to the facsimile reproduction by anyone of the patent document or the patent disclosure, as it appears in the Patent and Trademark Office patent file or records, but otherwise reserves all copyright rights whatsoever.

### REFERENCE TO SEQUENCE LISTING

The present application is being filed along with a Sequence Listing in electronic format. The Sequence Listing is provided as a file entitled 47CX-311983-US Sequence Listing, created Mar. 6, 2023, which is 27 kilobytes in size. The information in the electronic format of the Sequence Listing is incorporated herein by reference in its entirety.

### BACKGROUND

### Field

The present disclosure relates generally to the field of sequencing analysis, and more particularly to methylation sequencing analysis.

### Description of the Related Art

The goal of methylated alignment is to map converted (e.g., bisulfite treated) sequencing reads to their locations of origin on a reference genome; and then to determine if each cytosine on that location is methylated or unmethylated. To do this, four alignments for each read are typically performed—two different conversions of read bases, aligned against each of two different converted reference genomes. Then each base is analyzed to detect whether it was methylated, and ultimately gather statistics on the conversions to generate summary reports. There is a need for faster and/or more efficient methods for methylated alignment.

### SUMMARY

Disclosed herein include methods of methylation calling. The methods can be single-pass methylation calling methods. A single-pass methylation calling method can be at least 2× faster than a multi-pass methylation calling method. In some embodiments, a method for methylation calling is under control of a processor (e.g., a hardware processor or a virtual processor) and comprises: (a) receiving a reference genome sequence. The method can comprise: (b) generating a mapping reference sequence comprising a cytosine (C)-to-thymine (T) converted reference sequence and a guanine (G)-to-adenine (A) converted reference sequence from the reference genome sequence. The method can comprise: (c) receiving a plurality of sequence reads generated from a sample subjected to a methylation assay. The method can comprise: (d) for each of the plurality of sequence reads, (d1) generating a C-to-T converted sequence read and a G-to-A converted sequence read. The method can comprise: (d2) mapping (or aligning) the C-to-T converted sequence read and the G-to-A converted sequence to the mapping reference sequence to determine a converted sequence read mapped to the C-to-T converted reference sequence or the G-to-A converted reference sequence of the mapping reference sequence. The method can comprise: (e) determining (or calling), for each of a plurality of positions with Cs in the reference genome sequence, a number of the converted sequences that support methylated Cs and/or a number of the converted sequences that support unmethylated Cs at that position (e.g., for the sample) based on a number of Cs, if any, (which can indicate methylated Cs) and/or a number of Ts, if any, (which can indicate unmethylated Cs) of the converted sequences that are mapped to that position.

In some embodiments, a method of methylation calling is under control of a processor (e.g., a hardware processor or a virtual processor) and comprises: (ab) receiving a mapping reference sequence comprising a cytosine (C)-to-thymine (T) converted reference sequence and a guanine (G)-to-adenine (A) converted reference sequence generated from a reference genome sequence. The method can comprise: (c) receiving a plurality of sequence reads generated from a sample subjected to a methylation assay. The method can comprise: (d) for each of the plurality of sequence read, (d1) generating a C-to-T converted sequence read and a G-to-A converted sequence read. The method can comprise: (d2) mapping the C-to-T converted sequence read and the G-to-A converted sequence to the mapping reference sequence to determine a converted sequence read mapped to the C-to-T converted reference sequence or the G-to-A converted reference sequence of the mapping reference sequence. The method can comprise: (e) determining (or calling), for each of a plurality of positions with Cs in the reference genome sequence, a number of the converted sequences that support methylated Cs and/or a number of the converted sequences that support unmethylated Cs at that position (e.g., for the sample) based on a number of Cs, if any, and/or a number of Ts, if any, of the converted sequences that are mapped to that position.

In some embodiments, generating the mapping reference sequence comprises: indexing the C-to-T converted reference sequence and the G-to-A converted reference sequence as two separate reference sequences together. In some embodiments, the mapping reference comprises the C-to-T converted reference sequence and the G-to-A converted reference sequence with different reference identifiers. The C-to-T converted reference sequence can comprise all Cs in the reference genome sequence converted to Ts. The G-to-G converted reference sequence can comprise all Gs in the reference genome sequence converted to As.

In some embodiments, the C-to-T converted sequence read comprises all Cs in the sequence converted to Ts. The G-to-A converted sequence read comprises all Gs in the sequence converted to As. In some embodiments, the methylation assay comprises bisulfide sequencing, whole genome-bisulfide sequencing (WGBS), Enzymatic Methyl-seq (EM-seq), or TET-assisted pyridine borane sequencing (TAPS). The methylation assay can comprise a directional methylation protocol, a non-directional methylation protocol, or post-bisulfite adapter tagging (PBAT).

In some embodiments, the converted sequence mapped to the C-to-T converted reference sequence or the G-to-A converted reference sequence of the mapping reference sequence is the C-to-T converted sequence, a reverse complement of the C-to-T converted sequence, the G-to-A converted sequence, or a reverse complement of the G-to-A converted sequence. In some embodiments, mapping the C-to-T converted sequence read and the G-to-A converted sequence to the mapping reference sequence comprises: mapping the C-to-T converted sequence read and the G-to-A converted sequence to the mapping reference sequence to determine the best alignment amongst (i) the C-to-T converted sequence read and the C-to-T converted reference sequence, (ii) a reverse complement of the C-to-T converted sequence read and the C-to-T converted reference sequence, (iii) the G-to-A converted sequence read and the A-to-G converted reference sequence, and (iv) a reverse complement of the G-to-A converted sequence read and the G-to-A converted reference sequence. (i) The C-to-T converted sequence read, (ii) the reverse compliment of the C-to-T converted sequence read, (iii) the G-to-A converted sequence read, or (iv) the reverse compliment of the G-to-A converted sequence read resulting in the best alignment is the converted sequence read. The best alignment can have the highest alignment score. The best alignment can have no mismatch (or fewest mismatch(es)) in positions in the C-to-T converted reference genome or G-to-A converted reference genome corresponding to positions with Cs in the reference genome sequence to which (i) the C-to-T converted sequence, (ii) the reverse complement of the C-to-T converted sequence, (iii) the GC-to-A converted sequence, or (iv) the reverse complement of the G-to-A converted sequence is aligned. In some embodiments, mapping the C-to-T converted sequence read and the G-to-A converted sequence to the mapping reference sequence comprises: determining the converted sequence read is a top strand (OT), a reverse complement of the top strand (CTOT), a bottom strand (OB), or a reverse complement of the bottom strand (CTOB) based on alignment types.

In some embodiments, the number of the converted sequences that support methylated Cs at that position is the number of Cs, if any, of the converted sequences that are mapped to that position. The number of the converted sequences that support unmethylated Cs at that position can be the number of Ts, if any, of the converted sequences that are mapped to that position. Determining, for each of the plurality of positions with Cs in the reference genome sequence, a number of the converted sequences that support methylated Cs and/or a number of the converted sequences that support unmethylated Cs at that position can comprise: determining (or calling) (i) a percentage of the converted sequences that support methylated Cs and/or (ii) a percentage of the converted sequences that support methylated Cs using (i) a percentage of the converted sequences with Cs, if any, and/or (ii) a percentage of the converted sequences with Ts, if any, mapped to that position. The percentage of the converted sequences that support methylated Cs at that position can indicate the percentage of the DNA with methylated Cs at that position in the sample. The percentage of the converted sequences that support unmethylated Cs at that position can indicate the percentage of the DNA with unmethylated Cs at that position in the sample. In some embodiments, determining, for each of the plurality of positions with Cs in the reference genome sequence, the number of the converted sequences that support methylated Cs and/or the number of the converted sequences that support unmethylated Cs at that position comprises: determining (or calling), for each of one or more positions of the plurality of positions with Cs in the reference genome sequence, a number of the converted sequences that support C-to-T mutations based on a number of As of the converted sequences that are mapped to the complementary strand of the reference genome sequence at that position (and/or a number of Ts of the converted sequences that are mapped to that position). The number of the converted sequences that support C-to-T mutations at that position can be a number of As of the converted sequences that are mapped to the complementary strand of the reference genome sequence at that position (or the minimum of the number of As of the converted sequences that are mapped to the complementary strand of the reference genome sequence at that position and the number of As of the converted sequences that are mapped to the complementary strand of the reference genome sequence at that position). Determining, for each of one or more positions of the plurality of positions with Cs in the reference genome sequence, the number of the converted sequences that support C-to-T mutations can comprise: determining (or calling), for each of one or more positions of the plurality of positions with Cs in the reference genome sequence, a percentage of the converted sequences that support C-to-T mutations at that mutation using a percentage of the converted sequences that are mapped to the complementary strand of the reference genome sequence with As at that position. The percentage of the converted sequences that support C-to-T mutations at that position can indicate the percentage of DNA with C-to-T mutations at that position in the sample In some embodiments, the plurality of positions with Cs in the reference genome sequence comprises at least 10,000 positions, substantially all positions, or all positions with Cs in the reference genome sequence.

In some embodiments, the method further comprises: (d3) for each of one or more positions of the plurality of positions with Cs in the reference genome sequence to which a C or a T of the converted sequence read is mapped to, (i) increasing a counter of C in that position if the converted sequence read comprises a C mapped to that position; and (ii) increasing a counter of T in that position if the converted sequence comprises a T mapped to that position. a. In some embodiments, steps (d1) and (d2) are performed by an aligner. Steps (d1), (d2), and (d3) can be performed by an aligner.

In some embodiments, the method further comprises: creating a file or a report and/or generating a user interface (UI) comprising a UI element representing or comprising, for each of positions of the plurality of positions with Cs in the reference genome sequence, the number or percentage of the converted sequences that support methylated Cs and/or the number or percentage of the converted sequences that support unmethylated Cs at that position.

In some embodiments, the plurality of sequence reads comprises sequence reads that are about 100 base pairs to about 1000 base pairs in length each. The plurality of sequence reads can comprise paired-end sequence reads and/or single-end sequence reads. The plurality of sequence reads can be generated by whole genome sequencing (WGS), such as clinical WGS (cWGS). In some embodiments, the sample comprises cells, cell-free DNA, cell-free fetal DNA, amniotic fluid, a blood sample, a biopsy sample, or a combination thereof. The sample can be a sample of a subject. The sample can be obtained directly from a subject. The sample can be generated from another sample obtained from a subject. The other sample can be obtained directly from the subject.

Disclosed herein include systems for methylation calling. In some embodiments, a system for methylation calling comprises: non-transitory memory configured to store executable instructions. The non-transitory memory can be configured to store: a mapping reference sequence comprising a cytosine (C)-to-thymine (T) converted reference sequence and a guanine (G)-to-adenine (A) converted reference sequence generated from a reference sequence (e.g., a reference genome sequence, or a portion thereof). The system can comprise: a processor (e.g., a hardware processor or a virtual processor) in communication with the non-transitory memory, the hardware processor programmed by the executable instructions to perform: (a) receiving a plurality of sequence reads generated from a sample subjected to a methylation assay. The processor can be programmed by the executable instructions to perform: (b) for each of the plurality of sequence reads, (b1) generating a C-to-T converted sequence read and a G-to-A converted sequence read. The processor can be programmed by the executable instructions to perform: (b2) mapping the C-to-T converted sequence read and the G-to-A converted sequence to the mapping reference sequence to determine a converted sequence read mapped to the C-to-T converted reference sequence or the G-to-A converted reference sequence of the mapping reference sequence. The processor can be programmed by the executable instructions to perform: (c) determining (or calling), for each of a plurality of positions with Cs in the reference genome sequence, a number of the converted sequences that support methylated Cs and/or a number of the converted sequences that support unmethylated Cs at that position (e.g., for the sample) based on a number of Cs, if any, (which can indicate methylated Cs) and/or a number of Ts, if any, (which can indicate unmethylated Cs) of the converted sequences that are mapped to that position.

In some embodiments, the processor is further programmed by the executable instructions to perform: receiving the mapping reference sequence comprising the C-to-T converted reference sequence and a G-to-A converted reference sequence. The processor can be further programmed by the executable instructions to perform: generating the mapping reference sequence comprising the C-to-T converted reference sequence and a G-to-A converted reference sequence generated from the reference sequence. The mapping reference can comprise the C-to-T converted reference sequence and the G-to-A converted reference sequence with different reference identifiers. Generating the mapping reference sequence can comprise: indexing the C-to-T converted reference sequence and the G-to-A converted reference sequence as two separate reference sequences together. The C-to-T converted reference sequence can comprise all Cs in the reference sequence converted to Ts. The G-to-G converted reference sequence can comprise all Gs in the reference sequence converted to As

In some embodiments, the C-to-T converted sequence read comprises all Cs in the sequence converted to Ts. The G-to-A converted sequence read can comprise all Gs in the sequence converted to As. In some embodiments, the methylation assay comprises bisulfide sequencing, whole genome-bisulfide sequencing (WGBS), Enzymatic Methyl-seq (EM-seq), or TET-assisted pyridine borane sequencing (TAPS). The methylation assay can comprise a directional methylation protocol, a non-directional methylation protocol, or post-bisulfite adapter tagging (PBAT).

In some embodiments, the converted sequence is the C-to-T converted sequence, a reverse complement of the C-to-T converted sequence, the G-to-A converted sequence, or a reverse complement of the G-to-A converted sequence. In some embodiments, mapping the C-to-T converted sequence read and the G-to-A converted sequence to the mapping reference sequence comprises: mapping the C-to-T converted sequence read and the G-to-A converted sequence to the mapping reference sequence to determine the best alignment amongst (i) the C-to-T converted sequence read and the C-to-T converted reference sequence, (ii) a reverse complement of the C-to-T converted sequence read and the C-to-T converted reference sequence, (iii) the G-to-A converted sequence read and the A-to-G converted reference sequence, and (iv) a reverse complement of the G-to-A converted sequence read and the G-to-A converted reference sequence. The C-to-T converted sequence read, the reverse compliment of the C-to-T converted sequence read, the G-to-A converted sequence read, or the reverse compliment of the G-to-A converted sequence read resulting in the best alignment can be the converted sequence read. In some embodiments, mapping the C-to-T converted sequence read and the G-to-A converted sequence to the mapping reference sequence comprises: determining the converted sequence read is a top strand (OT), a reverse complement of the top strand (CTOT), a bottom strand (OB), or a reverse complement of the bottom strand (CTOB) based on alignment types.

In some embodiments, the number of the converted sequences that support methylated Cs at that position is the number of Cs, if any, of the converted sequences that are mapped to that position. The number of the converted sequences that support unmethylated Cs at that position can be the number of Ts, if any, of the converted sequences that are mapped to that position. Determining, for each of the plurality of positions with Cs in the reference genome sequence, a number of the converted sequences that support methylated Cs and/or a number of the converted sequences that support unmethylated Cs at that position can comprise: determining (or calling) (i) a percentage of the converted sequences that support methylated Cs and/or (ii) a percentage of the converted sequences that support methylated Cs using (i) a percentage of the converted sequences with Cs, if any, and/or (ii) a percentage of the converted sequences with Ts, if any, mapped to that position. The percentage of the converted sequences that support methylated Cs at that position can indicate the percentage of the DNA with methylated Cs at that position in the sample. The percentage of the converted sequences that support unmethylated Cs at that position can indicate the percentage of the DNA with unmethylated Cs at that position in the sample. In some embodiments, determining, for each of the plurality of positions with Cs in the reference genome sequence, the number of the converted sequences that support methylated Cs and/or the number of the converted sequences that support unmethylated Cs at that position comprises: determining (or calling), for each of one or more positions of the plurality of positions with Cs in the reference genome sequence, a number of the converted sequences that support C-to-T mutations based on a number of As of the converted sequences that are mapped to the complementary strand of the reference genome sequence at that position (and/or a number of Ts of the converted sequences that are mapped to that position). The number of the converted sequences that support C-to-T mutations at that position can be a number of As of the converted sequences that are mapped to the complementary strand of the reference genome sequence at that position (or the minimum of the number of As of the converted sequences that are mapped to the complementary strand of the reference genome sequence at that position and the number of As of the converted sequences that are mapped to the complementary strand of the reference genome sequence at that position). Determining, for each of one or more positions of the plurality of positions with Cs in the reference genome sequence, the number of the converted sequences that support C-to-T mutations can comprise: determining (or calling), for each of one or more positions of the plurality of positions with Cs in the reference genome sequence, a percentage of the converted sequences that support C-to-T mutations at that position using a percentage of the converted sequences that are mapped to the complementary strand of the reference genome sequence with As at that position. The percentage of the converted sequences that support C-to-T mutations at that position can indicate the percentage of DNA with C-to-T mutations at that position in the sample In some embodiments, the plurality of positions with Cs in the reference sequence comprises at least 10,000 positions, substantially all positions, or all positions with Cs in the reference sequence. In some embodiments, the system comprises a methylation caller which performs the step (c).

In some embodiments, the hardware processor is further programmed by the executable instructions to perform: (b3) for each of one or more positions of the plurality of positions with Cs in the reference sequence to which a C or a T of the converted sequence read is mapped to, (i) increasing a counter of C in that position if the converted sequence read comprises a C mapped to that position; and (ii) increasing a counter of T in that position if the converted sequence comprises a T mapped to that position. Determining, for each of the plurality of positions with Cs in the reference genome sequence, the number of the converted sequences that support methylated Cs and/or the number of the converted sequences that support unmethylated Cs at that position can comprise: determining, for each of the plurality of positions with Cs in the reference genome sequence, the number of the converted sequences that support methylated Cs and/or the number of the converted sequences that support unmethylated Cs at that position based on (i) a value of the counter for C at that position and (ii) a value of the counter for T at that position. In some embodiments, the system comprises an aligner which performs steps (b1) and (b2), and optionally (b3).

In some embodiments, the hardware processor is further programmed by the executable instructions to perform: creating a file or a report and/or generating a user interface (UI) comprising a UI element representing or comprising, for each of positions of the plurality of positions with Cs in the reference sequence, the number or percentage of the converted sequences that support methylated Cs and/or the number or percentage of the converted sequences that support unmethylated Cs at that position. The system can comprise an output module which creates the file or the report. The system can comprise a UI module which generates the UI element.

In some embodiments, the plurality of sequence reads comprises sequence reads that are about 100 base pairs to about 1000 base pairs in length each. The plurality of sequence reads can comprise paired-end sequence reads and/or single-end sequence reads. In some embodiments, the plurality of sequence reads is generated by whole genome sequencing (WGS), e.g., clinical WGS (cWGS).

In some embodiments, the sample comprises cells, cell-free DNA, cell-free fetal DNA, amniotic fluid, a blood sample, a biopsy sample, or a combination thereof. The sample can be a sample of a subject. The sample can be obtained directly from a subject. The sample can be generated from another sample obtained from a subject. The other sample can be obtained directly from the subject.

Also disclosed herein include a non-transitory computer-readable medium storing executable instructions, when executed by a system (e.g., a system with an FPGA), causes the system to perform any method or one or more steps of a method disclosed herein.

Details of one or more implementations of the subject matter described in this specification are set forth in the accompanying drawings and the description below. Other features, aspects, and advantages will become apparent from the description, the drawings, and the claims. Neither this summary nor the following detailed description purports to define or limit the scope of the inventive subject matter.

### BRIEF DESCRIPTION OF THE DRAWINGS

FIG. 1 depicts a diagram of the top applications for high-throughput analysis by input samples processed.

FIG. 2A-FIG. 2B depicts an exemplary multi-pass methylated alignment analysis.

FIG. 3 depicts an exemplary flowchart of the existing analysis pipeline.

FIG. 4 depicts an exemplary single-pass pipeline for single-end reads.

FIG. 5 depicts an exemplary illustration of the paired-end (PE) mapping solution.

FIG. 6 depicts an exemplary flowcharts of field programmable gate array (FPGA) analysis pipeline.

FIG. 7 depicts an exemplary embodiment of the methods disclosed herein, which includes loading in read hash table only once, and streaming the winner in second read.

FIG. 8 compares methylation multi-pass alignment (or mapping) methods to the presently disclosed single-pass alignment (or mapping) methods.

FIG. 9 shows an exemplary flowchart of the disclosed single-pass method with improvements in run time compared to existing multi-pass methods.

FIG. 10 depicts a non-limiting flowchart showing the overhead saving of the single-pass method compared to multi-pass mapping.

FIG. 11 shows non-limiting exemplary data related to time saving advantages (in hours) of the single-pass methods compared to existing methods.

FIG. 12A-FIG. 12D depict non-limiting exemplary methylation assays (directional assay illustrated in FIG. 12A-FIG. 12C; non-directional assay illustrated in FIG. 12D) and sequence reads generated. Methylation calling (e.g., single-pass or multi-pass methylation calling) can be used to process sequence reads generated using methylation assays.

FIG. 13 depicts an exemplary illustration of methylation alignment and strand determination.

FIG. 14 depicts an exemplary illustration of methylation calling which is downstream of methylation mapping. The methylation calling illustrated in in FIG. 14 can be implemented by single-pass and multi-pass methylation calling.

FIG. 15 shows an example of how C-to-T mutations are distinguished from bisulfate conversions.

FIG. 16 is a flow diagram showing an exemplary method of methylation alignment and calling.

FIG. 17 is a block diagram of an illustrative computing system configured to implement methylation calling.

Throughout the drawings, reference numbers may be re-used to indicate correspondence between referenced elements. The drawings are provided to illustrate example embodiments described herein and are not intended to limit the scope of the disclosure.

### DETAILED DESCRIPTION

In the following detailed description, reference is made to the accompanying drawings, which form a part hereof. In the drawings, similar symbols typically identify similar components, unless context dictates otherwise. The illustrative embodiments described in the detailed description, drawings, and claims are not meant to be limiting. Other embodiments may be utilized, and other changes may be made, without departing from the spirit or scope of the subject matter presented herein. It will be readily understood that the aspects of the present disclosure, as generally described herein, and illustrated in the Figures, can be arranged, substituted, combined, separated, and designed in a wide variety of different configurations, all of which are explicitly contemplated herein and made part of the disclosure herein.

All patents, published patent applications, other publications, and sequences from GenBank, and other databases referred to herein are incorporated by reference in their entirety with respect to the related technology.

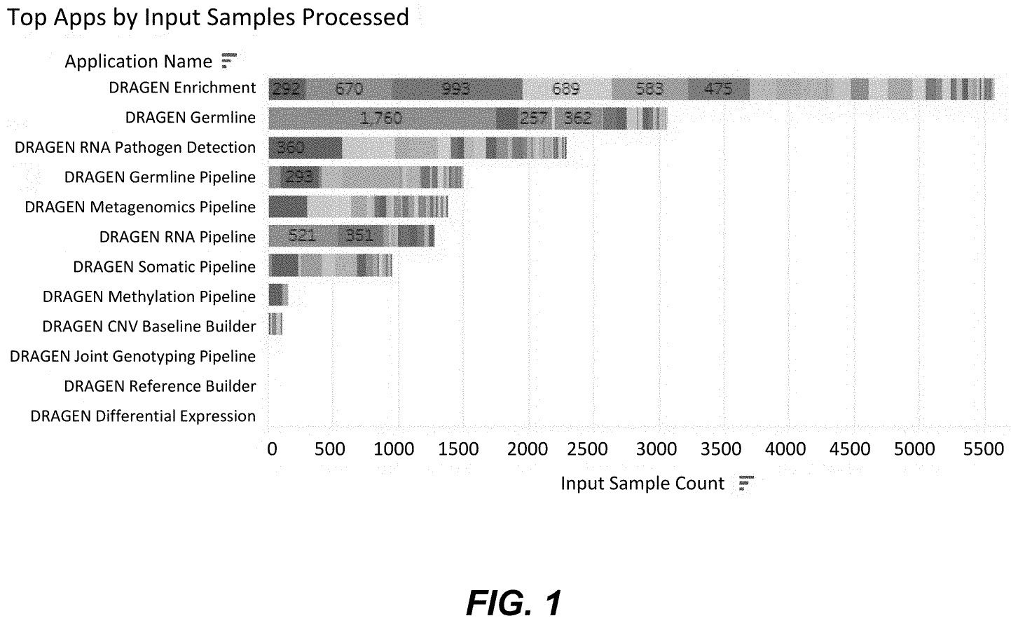

Many high throughput methylation sequencing analyses pipelines are slow, and use an enormous amount of disk space. Currently Illumina Dynamic Read Analysis for Genomics (DRAGEN) methylation requires 4-5 hours to complete on a Whole Genome Bisulfite Sequencing (WGBS) run, and on a Novaseq run with S2 flow cells that can fit 20 genomes, the DRAGEN methylation pipeline would take 3-4 days to complete analysis on the instrument. This speed makes computation the critical bottleneck. FIG. 1 shows the BaseSpace statistics of DRAGEN Methylation Application utilization as compared with the other applications.

Methylation sequencing is a significant and growing market. For example, GRAIL sequenced over 10,000 cell free DNA WGBS samples at 30× in their cohort and this need is most likely on-going for methylation bio marker discovery purposes. The methods described herein fill an unmet need for a high-throughput (e.g., Illumina DRAGEN) methylation pipeline on WGBS data (e.g., about 4-5 hours) that can run as fast as the DRAGEN Variant Caller (VC) pipeline on WGBS data (e.g., about 20 minutes), and can cache the hash from run-to-run to avoid reference re-loading overhead on panel sequencing.

Disclosed herein include methods of methylation calling. The methods can be single-pass methylation calling methods. A single-pass methylation calling method can be at least 2× faster than a multi-pass methylation calling method. In some embodiments, a method for methylation calling is under control of a processor (e.g., a hardware processor or a virtual processor) and comprises: (a) receiving a reference genome sequence. The method can comprise: (b) generating a mapping reference sequence comprising a cytosine (C)-to-thymine (T) converted reference sequence and a guanine (G)-to-adenine (A) converted reference sequence from the reference genome sequence. The method can comprise: (c) receiving a plurality of sequence reads generated from a sample subjected to a methylation assay. The method can comprise: (d) for each of the plurality of sequence reads, (d1) generating a C-to-T converted sequence read and a G-to-A converted sequence read. The method can comprise: (d2) mapping (or aligning) the C-to-T converted sequence read and the G-to-A converted sequence to the mapping reference sequence to determine a converted sequence read mapped to the C-to-T converted reference sequence or the G-to-A converted reference sequence of the mapping reference sequence. The method can comprise: (e) calling each of a plurality of positions with Cs in the reference genome sequence is a methylated C or an unmethylated C based on one or more Cs (which can indicate an methylated C) and/or one or more Ts (which can indicate an unmethylated C) of the converted sequences that are mapped to that position (or mapped to that position in the G-to-A converted reference sequence or in the C-to-T converted reference sequence.

Disclosed herein include systems for methylation calling. In some embodiments, a system for methylation calling comprises: non-transitory memory configured to store executable instructions. The non-transitory memory can be configured to store: a mapping reference sequence comprising a cytosine (C)-to-thymine (T) converted reference sequence and a guanine (G)-to-adenine (A) converted reference sequence generated from a reference sequence (e.g., a reference genome sequence, or a portion thereof). The system can comprise: a processor (e.g., a hardware processor or a virtual processor) in communication with the non-transitory memory, the hardware processor programmed by the executable instructions to perform: (a) receiving a plurality of sequence reads generated from a sample subjected to a methylation assay. The processor can be programmed by the executable instructions to perform: (b) for each of the plurality of sequence reads, (b1) generating a C-to-T converted sequence read and a G-to-A converted sequence read. The processor can be programmed by the executable instructions to perform: (b2) mapping the C-to-T converted sequence read and the G-to-A converted sequence to the mapping reference sequence to determine a converted sequence read mapped to the C-to-T converted reference sequence or the G-to-A converted reference sequence of the mapping reference sequence. The processor can be programmed by the executable instructions to perform: (c) calling each of a plurality of positions with Cs in the reference sequence is a methylated C or an unmethylated C based on one or more Cs and/or one or more Ts of the converted sequences that are mapped to that position.

### Methylation Sequencing Analysis

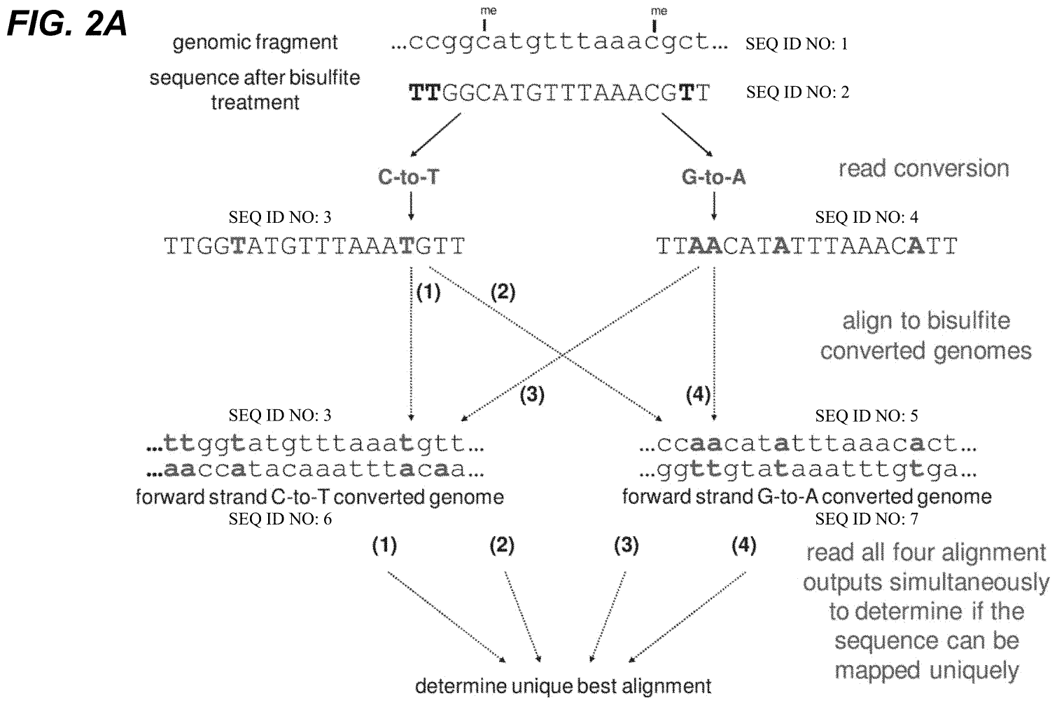

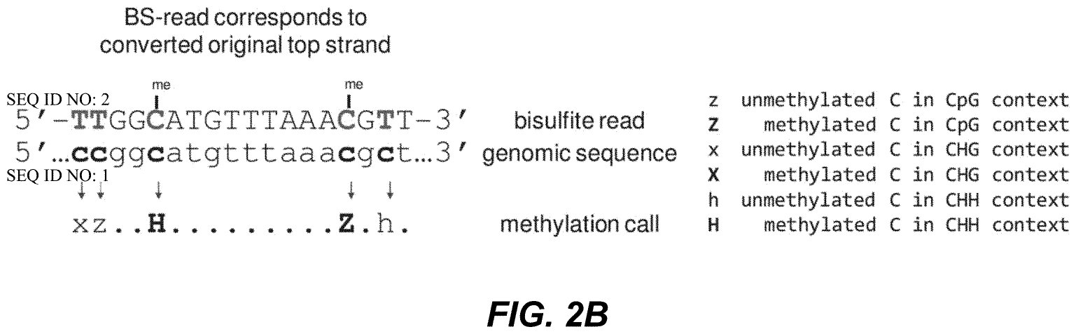

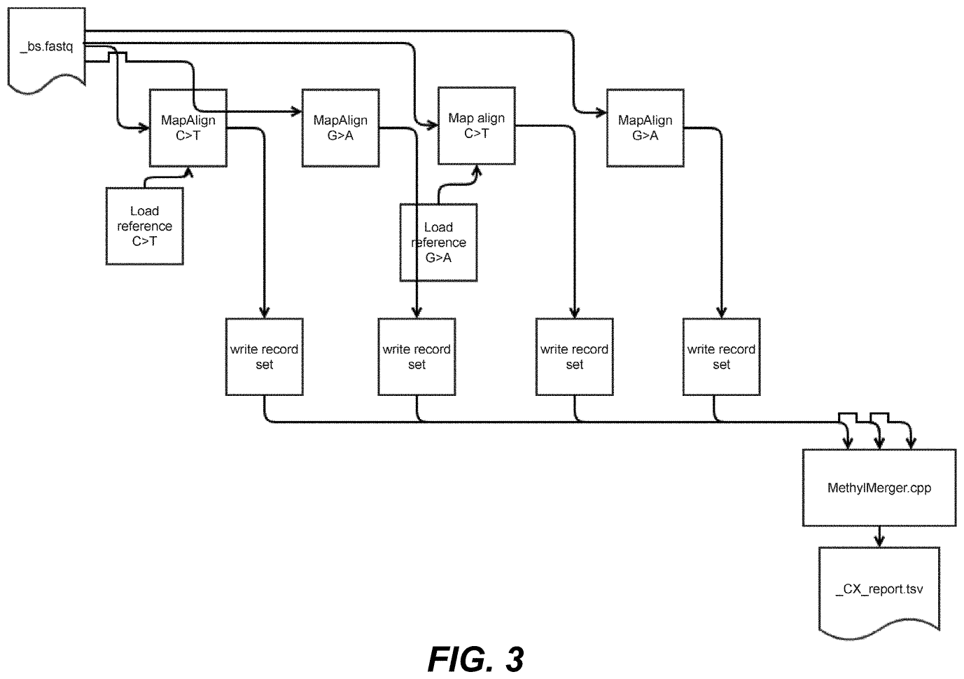

The goal of methylated alignment is to detect whether each cytosine position is methylated or unmethylated. To do this, four alignments for each read are typically performed—two different conversions of read bases, aligned against each of two different converted reference genomes. Then each base is analyzed to detect whether it was methylated, and ultimately gather statistics on the conversions to generate summary reports. This is diagrammed in FIG. 2A, in which reads from a BiSulfite-Sequencing (BS-Seq) experiment are converted into a C-to-T and a G-to-A version and are then aligned to equivalently converted versions of the reference genome. A unique best alignment is then determined from the four parallel alignment processes (in this example, the best alignment has no mismatches and comes from thread (1)). FIG. 2B shows the methylation state of positions involving cytosines is determined by comparing the read sequence with the corresponding genomic sequence. Depending on the strand a read mapped against this can involve looking for C-to-T (as shown here) or G-to-A substitutions. This means that for a typical run two different references are loaded, and four alignments per read are performed. So the runtime is more than 4× that of a normal, single-pass alignment run. Also, these alignments mostly spill to disk in uncompressed DBAM format, so disk consumption can be quite large. FIG. 3 shows how the existing Illumina Dynamic Read Analysis for Genomics (DRAGEN) pipeline works: DRAGEN methyl-call would conduct four separate DRAGEN map align runs, where each map align would output a record set and methyl_merger would then identify the best alignment per fragment. The intermediate recordset uses a disk space of 1 Tbyte for a Whole Genome Bisulfite Sequencing (WGBS) BAM, and determining the best alignment requires waiting on the final MapAlign to complete.

### Single Pass Methylation Pipeline

In some embodiments, an idea of the new design is to change the system to load a single compound hashtable, do the C>T and G>A read conversions on the fly, and choose the best alignment dynamically as the reads emerge from the aligner in parallel. Described below is an exemplary embodiment of the concept of single-end mapping and pair-ended (PE) mapping in this lean DRAGEN methylation method.

Building a Single Hash with Both C to T Converted and G to a Converted Genome

In some embodiments, this method builds a merged hash table using the native DRAGEN map align hash-building and a combined C>T/G>A FASTA. Given FASTA record chrExample with CCCCTTTT, it will convert into the following two FASTA records in the merged FASTA.

>chrExample_CT
TTTTGGGG
>chrExample_CT
CCCCAAAA

### Baseline Concept for Single Ended Read

Mapping and Counting:

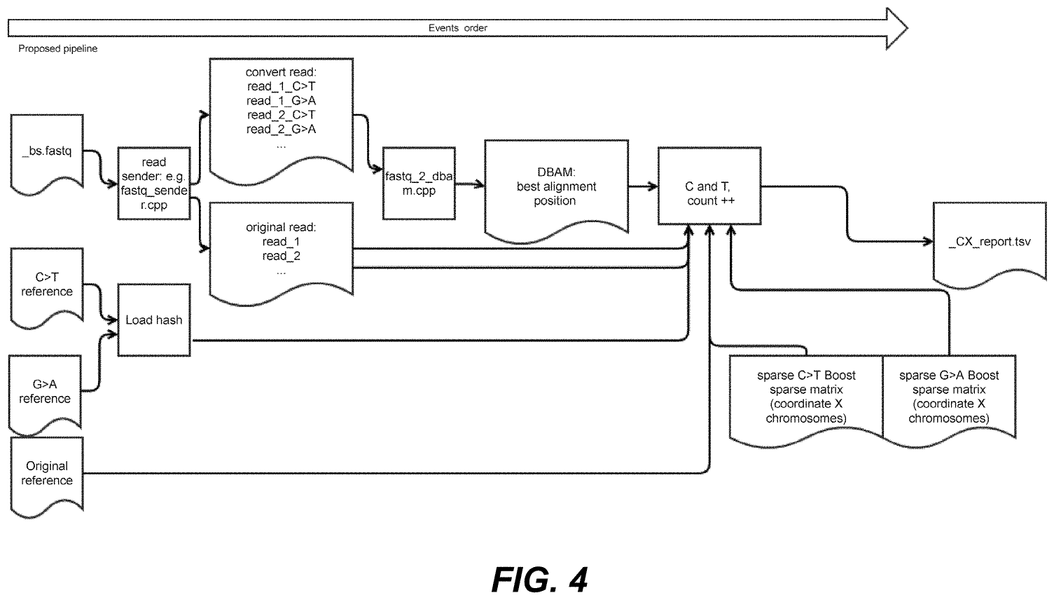

An exemplary illustration of the analysis pipeline for single-end reads is shown in FIG. 4. The input can comprise: (1) a single FASTQ, (2) a combined C>T/G>A reference hash, (3) the original reference sequence (and parameter: —preserve-alignment-order). The lean DRAGEN methylation method can comprise the following two data paths: (1) the lean DRAGEN methylation solution can convert each input FASTQ read record to a C>T converted record and a G>A record to avoid gaps from unmodified base from methylated Cs, (2) the read can be converted into DBAM records with read order preserved. Here are the corresponding code modifications: (1) modify FASTQ sender to tag and generate multiple converted versions of each read; (2) create logic that: catches the alignments, groups by fragment, chooses a winner, and emits only the winner alignment. SaTagGenerator type logic can be used as a template for grouping the FASTQ records. If one were to set —preserve-alignment-order, the converted read will be adjacent to each other.

This concept can be performed in Python. The concordance of C/T counts and site coverage between the present methods and prior methods is at 100% on chrM synthetic FASTQ.

### Pair Ended Read Support

Making a single hash table from the combination of two separate FASTAs can be challenging. Take the “directional” methylation protocol as an example, where two separate alignment runs are currently performed. In both runs, the read base-conversion process is the same. In run1, reads are aligned only against the C->T converted reference, and the allowable alignments are restricted to those where R1 is aligned in the forward direction and R2 is aligned to the reverse-complement. In run2, reads are aligned only against the G->A converted reference, and the allowable alignments are restricted to those where R2 is aligned in the forward direction and R1 is aligned to the reverse-complement. Presented herein is the following solution, where for a given fragment, said fragment will be converted into two fragments: one fragment with R1 C>T converted and R2 G>A converted, and one fragment with R1 G>A converted and R2 C>T converted.

```
R1:
  @Fragment0_R1_CT_GA
  <C to T converted sequence>
  +
  <qual string>
  @FragmentO_R1_GA_CT
  <G to A converted sequence>
  +
  <qual string>
  ....
R2:
  @Fragment0_R1 CT_GA
  <G to A converted sequence>
  <qual string>
  @Fragment0_R1 GA_CT
  <C to T converted sequence>
  +
  <qual string>
  ....
```

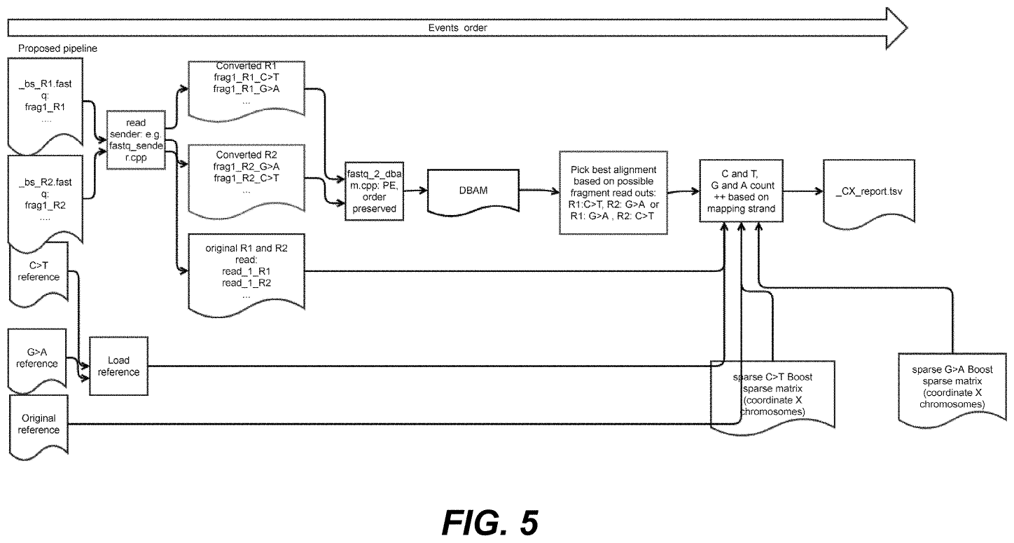

This paired end (PE) mapping approach results in: (1) 100% C/T and site coverage concordance when tested on a small synthetic dataset in both directional and non-directional mapping, and (2) 95% concordance in alignment in a difficult to map dataset, where 90% of the 5% disconcordant mapping can be explained by soft clipping. An exemplary illustration of paired-end mapping methods as disclosed herein is shown in FIG. 5.

### Pseudo Code of a Single-End Mapping: A Purely Streaming Based DRAGEN Methylation Solution

To simplify the illustration, only a single read is used here. In this example, the input can comprise:
(1) A single FASTQ with three sequences (one original read, one C>T converted, and one G>A converted) from a single read.
(2) a single Reference Genome with 1.) G>A, 2.) C>T; 3.) original sequences.
(3) optionally, a list of CpGs genomic locations.

The output can comprise:
(1) a hash table of CpG site by C and T counts in RAM, with 28 million CpG sites and with a counter for C and a counter for T per CpG site. The total memory consumption of a look up table chromosome per chromosome can be: (CT counts table+genomic position look up table per chromosome using search tree)*(4 bytes per int64)=(28*10\hat{ }6*2+28*log(28))*4=225 megabytes.
(2) Output a BAM.

Steps in MVP

In some embodiments, key steps include:
(1) Identify the best alignment start and end position within the reference from G>A, C>T.
(2) Extract the start and end position in the ReferenceGenome m_refgenome with the original sequence using MethylationMerger::extractReferenceSequence.
(3) Compare the read sequence with the extracted reference sequence and generate the methylation calls. In forward strand, walk forward and count the C that is a C in both the input read and the reference base. In reverse strand, walk backward and count the G that is a G in the reversed complemented reference. Here is the core logic:
   for each base,
if methylated:
   methylation_count[positon]+=1
if unmethylated:
   unmethylated_counts[position]+=1
(4) Export methylation_count and unmethylated_counts as a single TSV.

### Example Code Vectorization for Speed Up with Minimal Code Change

In some embodiments, the counting can be efficiently vectorized on a per read level to utilize the AVX vector instructions. Here, each read is treated as a vector of characters and the indexes with matching C-C's and matching C-Ts are found, where the reference C matches with either a C or T in the read. This approach has the advantage of simplifying the code, (i.e. collapsing a for-loop as a single boost vector instruction) and utilizes the SIMD hardwares that handle vector operations on CPU via C++ Boost library without the need of extra coding. In some embodiments, if, for example, the counting throughput is still not high enough, the bytes can be packed together for some instructions with logical operators to replace some of the following instructions before pthreads can be utilized for parallelizing the loop, which could, in some embodiments, potentially be more complicated and generate limited speed up for inner-loop optimizations. The following pseudo code shows how vector instructions can be used to append the C/T counts by treating each read as a vector:

//constant parameters
max_length=200; //to be determined
read_base_positions=[0....max_length] //
Boost::Map methylated_c_count_sparse_matrix; //chr X coordinate matrix, each entry
contain the C base counts from input reads
Boost::Map unmethlyated_c_count_sparse_matrix; //chr X coordinate matrix, each entry
contain the T base counts from input reads
//input
input_seq; //ATCGGTT
best_mapping_chr;
best_mapping_coordinate;
//load refseq of mapped coordinate
ref_seq=load_ref_sequence(best_mapping_coordinate)//ACCGGTT
ref_seq_v=boost: dense_vector (ref_seq) //ACCGGTT
ref_seq_c_mask = (ref_seq_v==“C”) //0110000
input_seq_c_mask=(input_seq==“C”) //0010000
input_methylated_c_mask=input_seq_c_mask==ref_seq_c_mask //0010000
input_seq_t_mask=(input_seq==“T”) //0001000
input_unmethylated_c_mask=input_seq_c_mask==ref_seq_c_mask //0100000
sparse_index_to_append_methlyated_c=pointwise_multiplication(input_methylated_c_mask,
read_base_positions_v); //0020000
methylated_c_count_sparse_matrix[best_mapping_chr,sparse_index]+=1 ;
sparse_index_to_append_unmethlyated_c=pointwise_multiplication(input_unmethylated_c_
mask,read_base_positions_v); //0100000
methylated_c_count_sparse_matrix[best_mapping_chr,sparse_index]+=1;

### Run Time Scaling

A major difference between the present method and the existing methods (e.g., DRAGEN multi-pass methylation design) is the method as disclosed herein can advantageously run methylation analysis in one map align run, while streaming the read and appending the methylated and unmethylated C count directly in memory. Non-limiting examples of advantages of the presently disclosed single-pass methylation solution over existing methods include: (1) Run times can be as fast as just running 2 map alignments natively with minimal components and code; (2) Non-limiting advantages that a one pass DRAGEN methylation run yield include (a) Loading the DRAGEN reference hash table only once, eliminating the need for reloading reference 4 times and utilizing genome caching from run to run, (b) Better integration with the rest of the components without a separate workflow to re-initialize map alignment 4 times; (3) Operate reads in a purely streaming fashion and eliminate the need of 4 intermediate DBAM write and 4 DBAM read passes from disk, which in turn eliminates the current high disk intermediate storage requirement (1 TB) and improves speed; (4) Simplifies the plug-in with other high throughput analysis components: e.g., deduplication, sorting, and VC calls on non-methylation sites.

A speed-up of 6× is estimated based on the following results, with a 4× speed by combining the references and just count, and a 2-3× MapAlign throughput increase by optimizing the FASTQ read conversion code.

### Additional Features

Additional embodiments can include masking the C/G with N (a wild card base) in in-silico bisulfite converted reads to map onto the bisulfite converted genome. Without being bound by any particular theory, this can reduce mapping accuracy but reduce code complexity at the same time, as a majority of the C will become wild card in mapping in hyper-methylated reads.

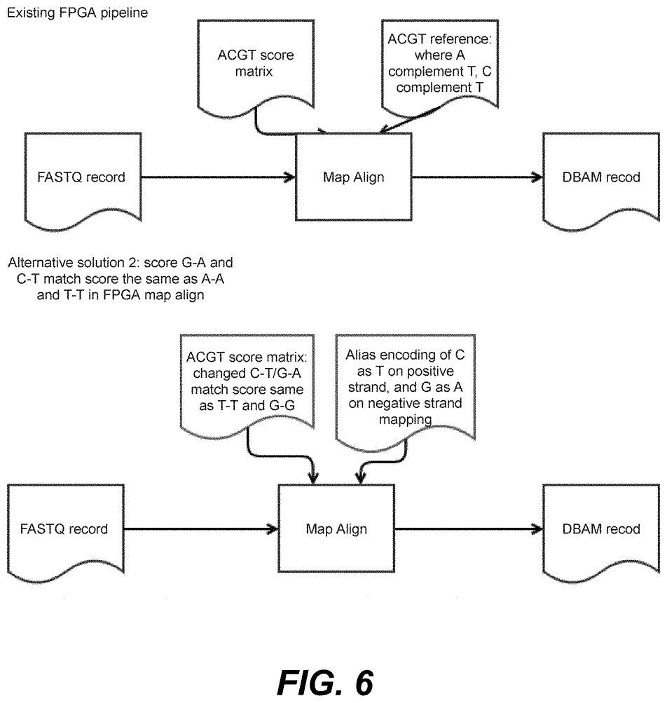

Another embodiment can include field programmable gate array (FPGA) code change: If DRAGEN map align uses a scoring matrix to calculate the mismatch penalty score, the FPGA map align can be configured such that C-T base matches would have the same behavior as T-T matches, and G-A the same as A-A in two separate runs. The output read sequence would preserve the input read identity, and the native loaded genome can be compared against the read mapped location. T should match with T if DRAGEN map align uses a scoring matrix to calculate the mismatch penalty score, the FPGA Map Align can be configured such that C-T base matches would have the same behavior as T-T matches to mimic the C to T read conversion, and G-A the same as A-A to mimic the G to A read conversion. In some embodiments, the analysis may need to interleave the one C to T converted and one G to A converted read, as those reads cannot be treated the same. In some embodiments, this solution would require swapping in-and-out out the score for C-T penalty and G-A penalty to interleave the reads on FPGA (See, FIG. 6).

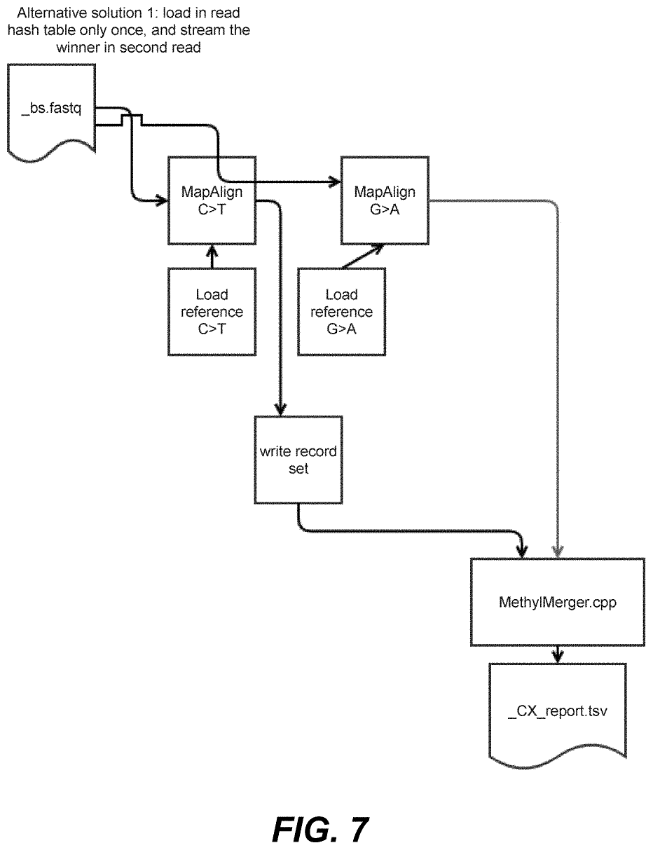

In another embodiment, the method can include loading in a read hash table once and streaming the winner in the second pass. The two hashtable logic is kept, but alignments are not spilled that can already be determined are not the best (Same general logic as is currently implemented). Two reference hashtables are loaded one after the other, one C→T converted, the other G→A converted. The difference is that when the first hashtable is loaded, all the base conversions and alignments for each read that use that hashtable (1 or 2 alignments depending on directional/non-directional) are performed, the winner is picked, and only the winner is written to an intermediate DBAM. When the second hashtable is loaded, all the information needed to pick the overall winner for each read is available. Again, all the base conversions and alignments for each read that rely on the second hashtable are performed, and the winner is picked from these alignments and the winning alignment from the previous hashtable that has been stored. This overall winner is then sent downstream. This can advantageously allow for only one intermediate DBAM as opposed to two or four (See, FIG. 7). This may not improve runtime if the spills are not a speed bottleneck and requires two distinct reference hashtables.

In another embodiment, the existing methyl merger code can be parallelized in C++.

TABLE 1
Exemplary Characteristics of Methylation Calling Methods

| | | | Parallelize | |
|---|---|---|---|---|
| | | | based on | |
| | | | existing | Two runs |
| | Disclosed solution | Existing solutions | solutions | approach |
| Characteristics | 1. Better native integration | 1. Using existing | 1. Can reuse | 1. Quick to |
| | with the other components. | solution means no | most of the | implement |
| | One run of map align without | code change. | code while | given |
| | board resetting, enabling | 2. Already enables | achieving | similarly to |
| | downstream component to | a sort and de-dup | speed up. | current |
| | stream the reads also, e.g. | solution. | | logic. |
| | dedup, sorting, VC on non- | 3. Slow and takes | | 2. Potentially |
| | cpg sites, on target bed file | up TB worth of | | speed up by |
| | region. | intermediate storage | | halving the |
| | 2. Achieve a speed up of 4.5 | for WGBS data | | amount of |
| | fold in WGBS data, and | processing. | | reads that |
| | finish in ~30 minutes, | | | need to |
| | enable on instrument usage. | | | write/read |
| | 3. Final results would be | | | on disk. |
| | highly concordant with the | | | |
| | existing methods. | | | |
| | 4. Simplify the methylation | | | |
| | codebase which would benefit | | | |
| | clinical research use | | | |
| | authorization. | | | |
| | 5. Easier for parallelization | | | |
| | when starting from a small | | | |
| | amount of code. | | | |
| | 6. Enable hash caching from | | | |
| | run to run: important for | | | |
| | small sample. | | | |
| | 7. A bigger hash table | | | |
| | (e.g., 3X). | | | |

In some embodiments, the bisulfite reads can be input into DRAGEN multiple times natively in the FASTQ sender. In some embodiments, methylation BAM files can be generated by adding a filter in BAM using MethylMerger::emitPair and taking only the original alignment based on the added feature in QNAME. If unique molecular identifiers (UMI) are needed, a bit vector can be used to keep track of the UMI seen so far per CpG position. 6 digit UMIs only require 12 bits per CpG, which is still reasonable in the total memory size.

### Single-Pass Methylation Mapping

Existing methylation-calling pipelines require two or four mapping passes over all the reads, with two different adjusted references, and various settings for conversion of read bases and restricted alignment orientations. All reads are mapped to 2-4 DBAM files on disk, then these are read back in and the 2-4 alignments of each read are merged into a final alignment. This is a slow process due to the multiple mapping passes and the large amount of file input/output.

Described herein is a method to effectively and advantageously execute the 2-4 alignments and their merge in a single mapping pass. In some embodiments, the two adjusted references are combined into a single hash table, and the mapper automatically tries all the desired alignment options, selects and returns the best one. In some embodiments, the single mapping pass can run somewhat slower than each legacy pass since it is iterating over more possibilities, but the whole operation can be dramatically faster.

### Mapping for Methylation Calling

Provided herein is an overview of bisulfite-treated methyl-seq protocols, and methods for mapping reads and calling methylation status.

Depending on the exact library protocol, read alignments with certain combinations of three factors can be considered: (1) Reference base conversions (C→T or G→A), (2) Read base conversions (C→T or G→A), and (3) Alignment orientation allowed (forward or reverse-complemented (RC)). Without being bound by any particular theory, since the two mates in paired-end libraries are read in opposite directions, the latter two factors (read conversion and alignment orientation) always have the opposite settings for the two mates. Below is a table (Table 2) showing the alignment types required by three different methyl-seq protocols:

TABLE 2
Alignment Types

| | | | Read | Read | Orientation |
|---|---|---|---|---|---|
| Protocol | BAM | Reference | 1 | 2 | Constraint (R1) |
| directional | | | | | |
| | 1 | C->T | C->T | G->A | Forward-only |
| | 2 | G->A | C->T | G->A | RC-only |
| non-directional, or | | | | | |
| directional-complement | | | | | |
| | 1 | C->T | C->T | G->A | Forward-only |
| | 2 | G->A | C->T | G->A | RC-only |
| | 3 | C->T | G->A | C->T | RC-only |
| | 4 | G->A | G->A | C->T | Forward-only |
| PBAT | | | | | |
| | 3 | C->T | G->A | C->T | RC-only |
| | 4 | G->A | G->A | C->T | Forward-only |

### Reference Construction

In some embodiments, a single mapping reference is built (hash table, reference.bin, etc.) containingbothC→T and G→A conversions of the original (FASTA) reference sequences. Each reference sequence can have two copies included, with consecutive REF_ID indices.
Even REF_ID=2N+0: C→T conversion of sequence N
Odd REF_ID=2N+1: G→A conversion of sequence N
   Reference.bin can be accordingly twice as long as usual. The hash table can be constructed to index all of these sequences (both conversions), as if they are unrelated sequences. Due to the double-sized reference, seeds can be populated in the hash table with reduced (˜50%) density.

Another embodiment, the “multi-base method,” may have accuracy advantages. This is similar to the above construction, but: (1) In reference.bin, the C→T and G→A conversions are not done by replacing nucleotides, but rather by using a two-base code matching both nucleotides, the substitutions are thus C→Y=[CT] and G→R=[AG]; (2) straight C→T and G→A conversions, not multi-base codes, are still used when building the reference buffer used for hash table construction.

Also note that using seed length (ht-seed-len) 27 instead of the default 21 can be important for methylation alignment. Without being bound by any particular theory, this is because, with the C→T or G→A base conversions, around 20% of the entropy (information content) of DNA sequences is destroyed, so longer seeds are required to expect reasonably unique mapping. Seed length 21 results in some noise matches that can slow down the mapper. In some embodiments, the default is changed to 27 when methylation modes are enabled.

### Configuration Registers

Table 3 shows the Map/Align configuration registers for Single-Pass Methylation Mapping, referenced by name herein.

TABLE 3
Configuration Registers

| Address | Module | Name | Format | Description |
|---|---|---|---|---|
| 0x80358 | Mapper | methyl_map_mode | 2-bit | 0 = Normal (non-methyl-seq or legacy |
| | | | unsigned | operation) |
| | | | | 1 = Seed mapping converts R1 C→T, |
| | | | | R2 G→A |
| | | | | 2 = Seed mapping converts R1 G→A, |
| | | | | R2 C→T |
| | | | | 3 = Seed mapping tries both C→T and |
| | | | | G→A conversions, for each mate |
| 0xC0388 | Aligner | methyl_aln_mode | 2-bit | Bit 0: Convert read bases for alignment |
| | | | vector | Bit 1: Restrict alignment orientations |

### Configuration Values

In some embodiments, user parameters are somewhat different from the hardware parameters above. In some embodiments, the plan for single-pass methylation mapping is: (1) methyl_map_mode set according to the library protocol (1 for directional, 2 for PBAT, 3 for non-directional, directional-complement), (2) methyl_aln_mode=3, enabling both read base conversion and orientation restrictions. If the “multi-base method” is used, then instead: methyl_aln_mode=2, not converting read bases for alignment, but still restricting orientations. In some embodiments, all these parameters should be zeros when not running single-pass methylation mapping.

Seed Editing

In some embodiments, seed editing (Mapper.edit-mode >0) is not supported along with single-pass methylation mapping and if a user parameters combine these, software can either fail, or warn and force seed mapping off.

Query Seed Density

In some embodiments, because reference seeds are populated with only ˜50% density to accommodate the double reference, seeds can be queried with 100% density (Mapper.seed-density=1) rather than the default 50%, to avoid sensitivity loss. In some embodiments, retain 50% query seed density is retained.

Read Trimming

Except on “Eagle” systems, the mapper has read trimming capabilities, which can support the best practices for methylation calling: (1) Adapter trimming and (2) Bisulfite trimming (trimming an extra 2 bp from either end depending on sequence content and adapter detection).

In some embodiments, when read trimming is enabled, there can be trimming to very few or zero bases. Rather than omitting trivial or empty reads, too-short reads can be “filtered,” they can be replaced with “ ” sequences (to pick length 10). This can be done by configuring read filter settings min-read-len (e.g., 11) and filter-dummy-len (e.g., 10). In some embodiments, such settings can be default, so any time one enables read trimming options, this avoids empty reads. In some embodiments, the filter-set-flag is set to the “disqualified” SAM flag 0x200 on such filtered reads.

Additional Settings

In some embodiments, themap_orientationsregister used for legacy methylation mapping can be set to zero. Orientations restrictions can vary depending on each alignment candidate's reference and read conversions. Other map/align settings may be modified as usual for methylation mapping, e.g. global=1, no-unpaired=1, etc.

### Behavior

Seed Mapping

In some embodiments, the seed mapping stage will query the hash table with read bases converted according to methyl_map_mode. When methyl_map_mode=3, seeds can be mapped using both C→T and G→A conversion options. In some embodiments, this iteration will be implemented as an inner loop, generating both versions at each seed position.

Seed Chaining

In some embodiments, all seed hits will be grouped into seed chains, potentially from both reverence conversions (even and odd REF IDs), both read conversions, and both orientations. Each seed chain already accepts seeds of only a single orientation, and near each other in the reference so there is only one reference conversion. In some embodiments, each seed chain must only accept seed hits from one read conversion (C→T or G→A). A flag indicating which read conversion can be saved in the seed chain record, and propagated through the map/align pipeline.

Paired End Processing

In some embodiments, mate seed chains with the same read conversion will not be considered proper pairs. In some embodiments, triggered rescue scans will be flagged with the opposite read conversion.

Alignment

All alignment-like operations (rescue scan, gapless alignment, Smith-Waterman) can operate with the same adjustment. In some embodiments, if methyl_aln_mode bit #0 is set, read bases will be converted either C→T or G→A, as flagged in the seed chain record (Conversion on-the-fly by hardware).

In some embodiments, e.g., the “multi-base method,” read bases are not converted for alignment. Rather, the reference sequence has multi-base codes, able to match both the original and (seed-mapping) converted base.

Orientation Restriction

In some embodiments, if methyl_aln_mode bit #1 is set, illegal combinations of reference conversion, read conversion, and alignment orientation must be discarded. In existing methylation mapping, alignment orientation was restricted very early in the pipeline, by dropping seed hits with the wrong orientation.

All protocols allow read alignments of both orientations, depending on the combination of reference conversion and read conversion. But due to the global coordinate system, it can be unknown which reference contig alignments are inside until late in the pipeline, after alignment operations, when ref_index.bin is queried. Only at that stage can one discover the even or odd REF_ID, which implies the reference conversion, and determine whether a given alignment candidate has legal orientation.

The legal combinations are:
Reference C→T (even REF_ID), read C→T, aligned forward
Reference C→T (even REF_ID), read G→A, aligned RC
Reference G→A (odd REF_ID), read C→T, aligned RC
Reference G→A (odd REF_ID), read G→A, aligned forward

This can be expressed as a simple Boolean logic requirement: (REF_ID & 1) XOR readG2A XOR alignedRC=0

These restrictions apply identically for both mates. (Later, to be properly paired, mate alignments can be to the same REF_ID with opposite orientation.) In some embodiments of the presently disclosed method, any alignment with an illegal orientation (given reference and read conversions) will be discarded immediately after the reference index lookup.

Alignment Selection and Output

In some embodiments, there are no changes in the operation of alignment comparison, MAPQ calculation, or engine output as compared to existing methods. The winning alignment is chosen by best pair score, regardless of type, and all alignments compete normally for MAPQ. In some embodiments, changes can be made to more closely model current software's 2-4 alignment merge operation.

In some embodiments, alignments to the same position in the even and odd versions of the same contig do not compete for MAPQ purposes. In some embodiments, alignments do compete. Without being bound by any particular theory, methylation calling may need confidence on which strand was bisulfite-treated just as much as confidence in alignment position. In some embodiments, pairs are forced unmapped if two alignment candidates score equally well, or within some tolerance. In some embodiments, it can be assumed that software can react to MAPQ for this purpose. In some embodiments, one alignment type is prioritized over another on ties.

Software can determine the alignment type information as follows: (1) Alignment direction indicated by FLAGs, (2) Reference conversion implied by REF_ID (even or odd), and (3) Read conversion is the one which is legal for this combination of reference conversion and alignment direction (readG2A=(REF_ID & 1) A ((FLAG>>4) & 1)).

NM, AS, and XS Tags

Alignment scores (AS & XS) and mismatch counts (NM) can be calculated based on alignments with both reference and query bases converted. In some embodiments, AS and XS are left alone; they reflect the appropriate underlying scoring scheme. The final BAM output contains the original read sequences, and is officially relative to the standard, unconverted reference. So, in some embodiments, the mapper's NM values will be officially incorrect. While, in some embodiments, it seems less likely that tools are going to rely on NM tags in methylation analysis pipelines, NM is a very standard tag. Therefore, a NM recalculation for “graph reference” support can be applied here.

### Methylation Single Pass Method

FIG. 2A-FIG. 2B show exemplary diagrams of existing methylation mappers/aligners. As shown in FIG. 2A, existing mappers typically require aligning reads to reference up to 4 times (CT/GA converted reads to CT/GA converted reference). FIG. 2B shows that alignment results are passed to a methylation caller which determines the best alignment for methylation caller.

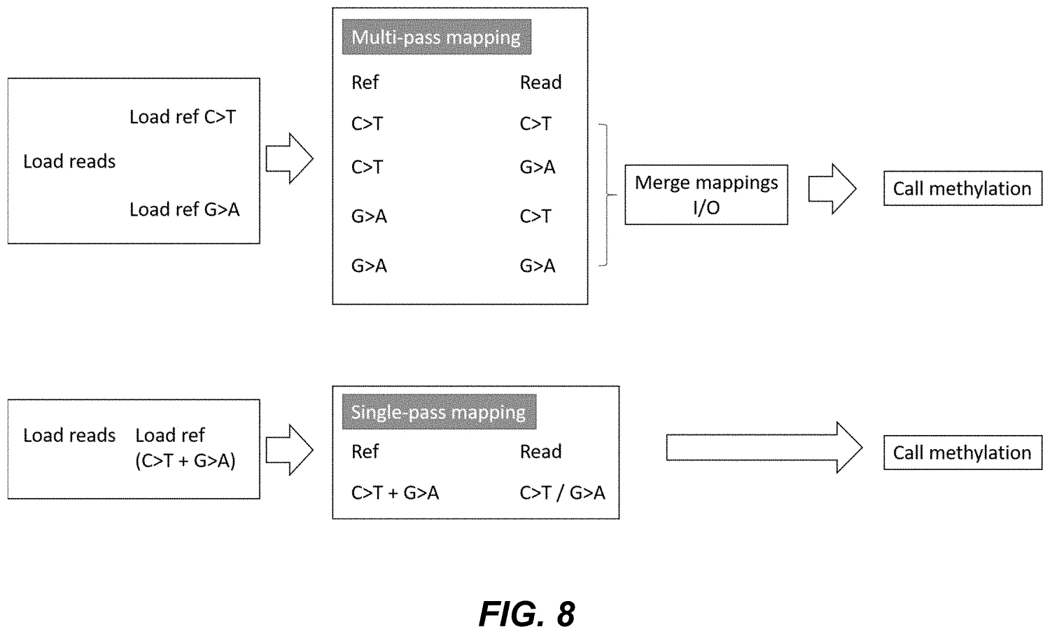

FIG. 8 depicts exemplary flowcharts comparing the present methods with existing methods. The presently disclosed single-pass method makes the CT/GA conversion during alignment, passing the reads one single time, (e.g., single-pass). This saves the alignment runtime up to, in some embodiments, 4 fold.

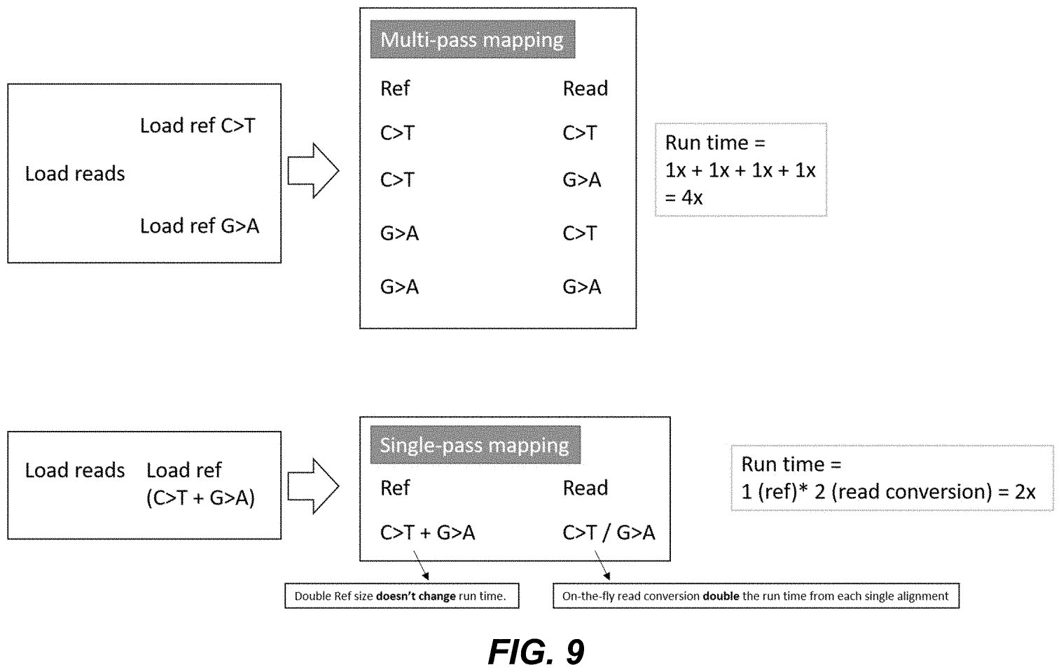

FIG. 9 depicts a non-limiting exemplary flowchart related to increased mapping speed of the present method. The most time-saving step takes place at mapping. DRAGEN uses a similar mapping strategy as the most widely used mapping algorithms (e.g., Bowtie, BWA), which all involve indexing to a reference genome. Interestingly, the size of the genome does not impact the runtime of mapping. For example, mapping a set of reads to a genome of N bp takes essentially the same time as mapping to a genome of 2*N bp. Therefore, by pre-building combined genome index with both C->T and G>A conversion, the mapping time can be shortened by 2 fold.

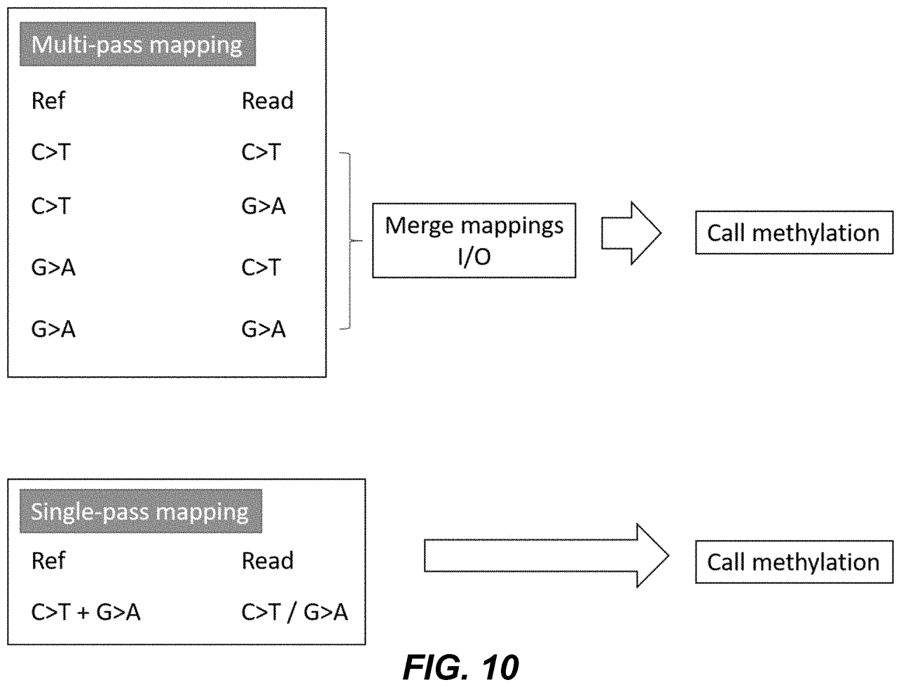

Overhead savings are depicted in FIG. 10. There are various overheads incurred by multiple mapping passes. This can include input/output to and from memory to disk=(I/O), merging mappings, and making request to aligners. Those overheads happen up to four times during multi-pass, but one time during single-pass. There are other factors contributing to runtime improvement. For example, during mapping, converting reads by C>T and G>A and query the aligner one time can be faster than mapping the reads two times. Ultimately, during real-world data test, a total three times run time improvement can be observed.

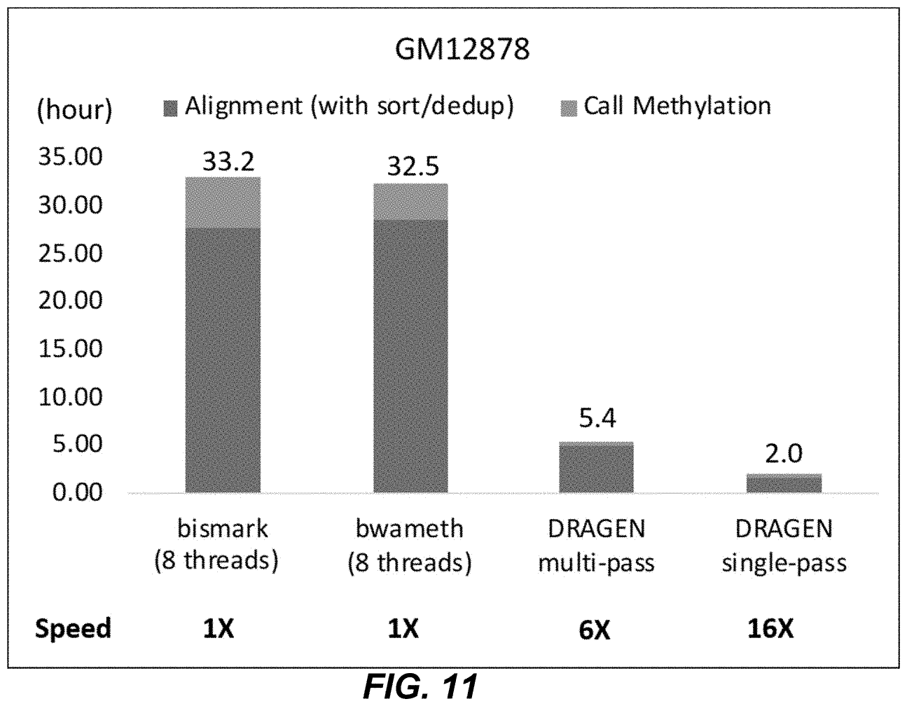

A single-pass improves runtime compared to existing methods on, for example, GM12878 cells (FIG. 11): (1) 3× faster than DRAGEN multi-pass and (2) 16× faster than bismark/bwameth. The present single pass method can perform a typical 30× WGBS analysis in <2 hours.

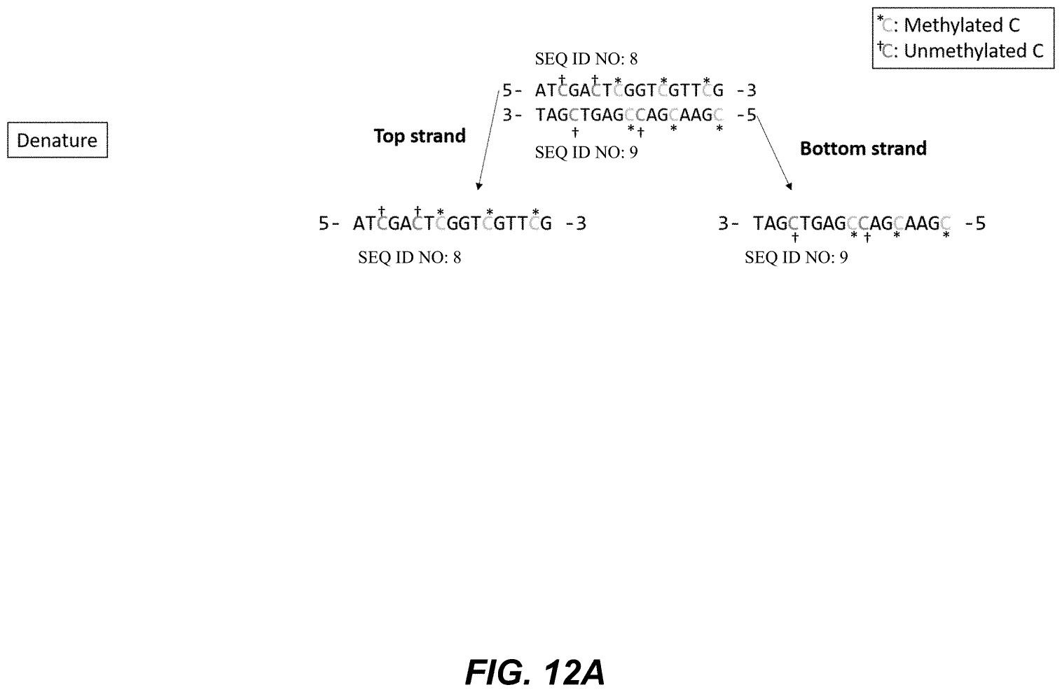

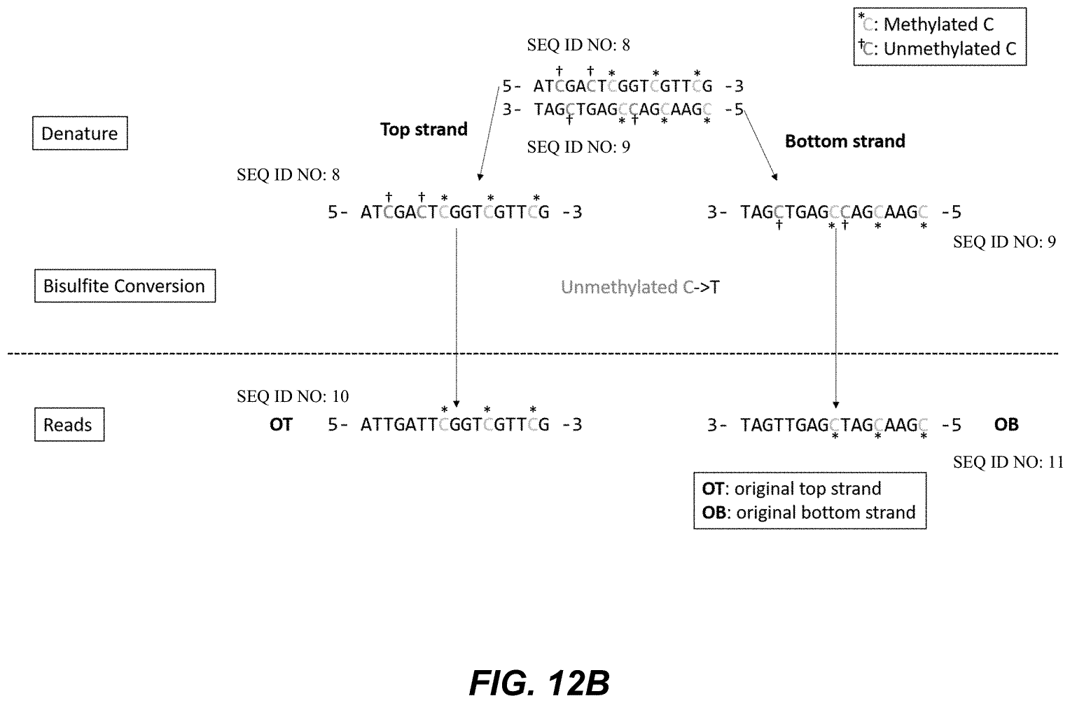

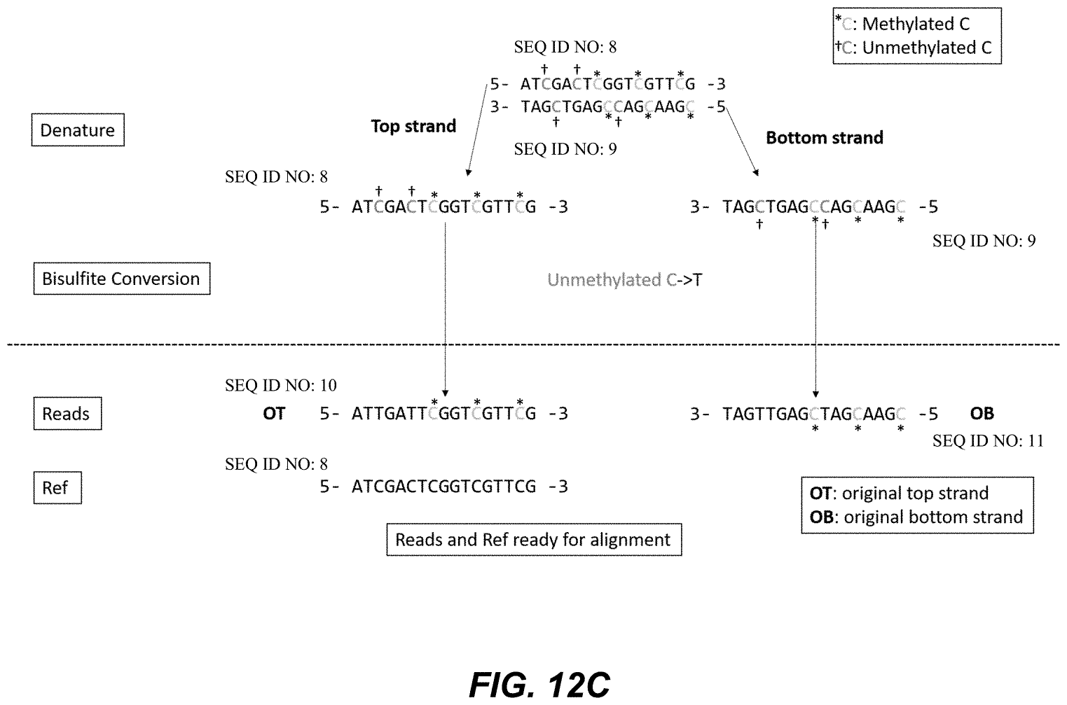

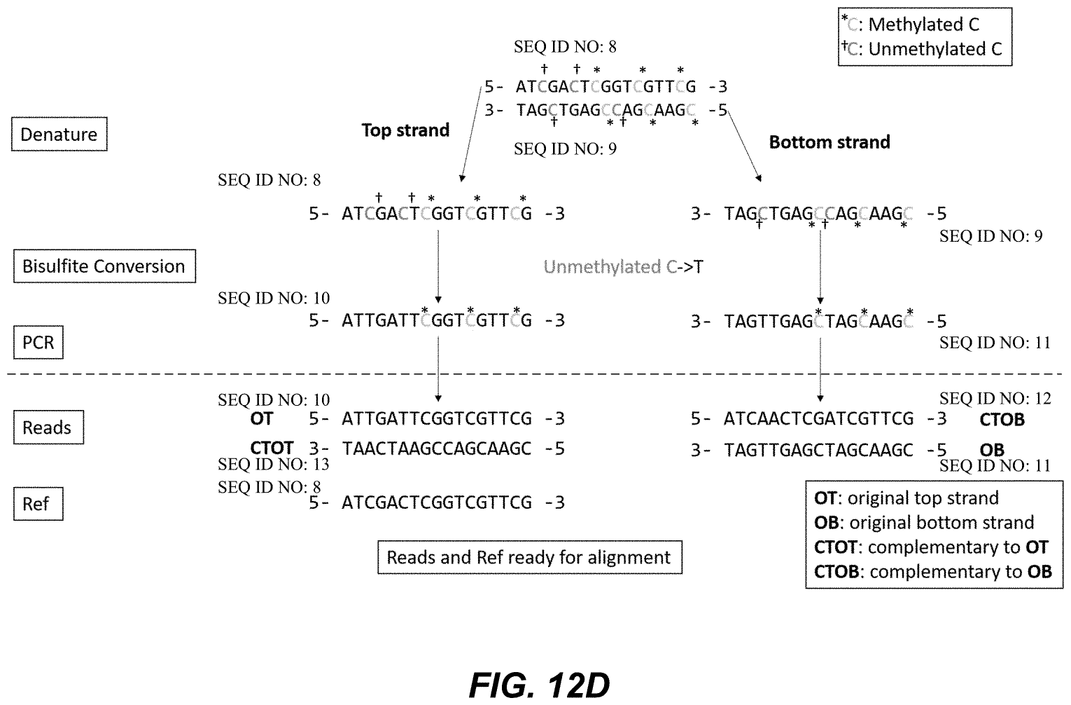

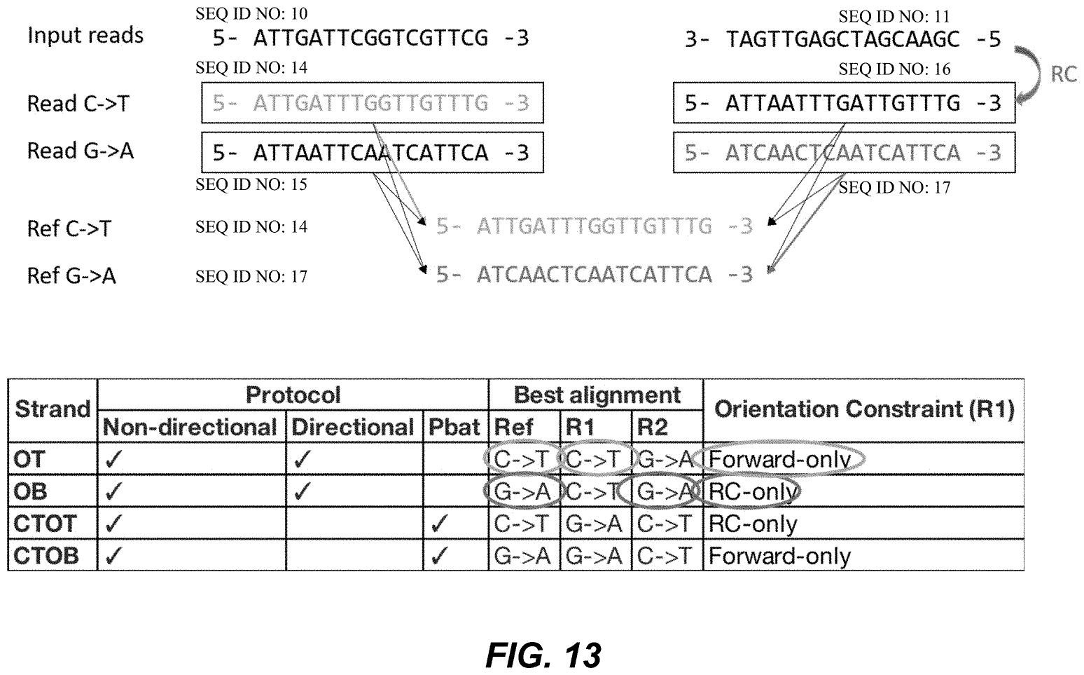

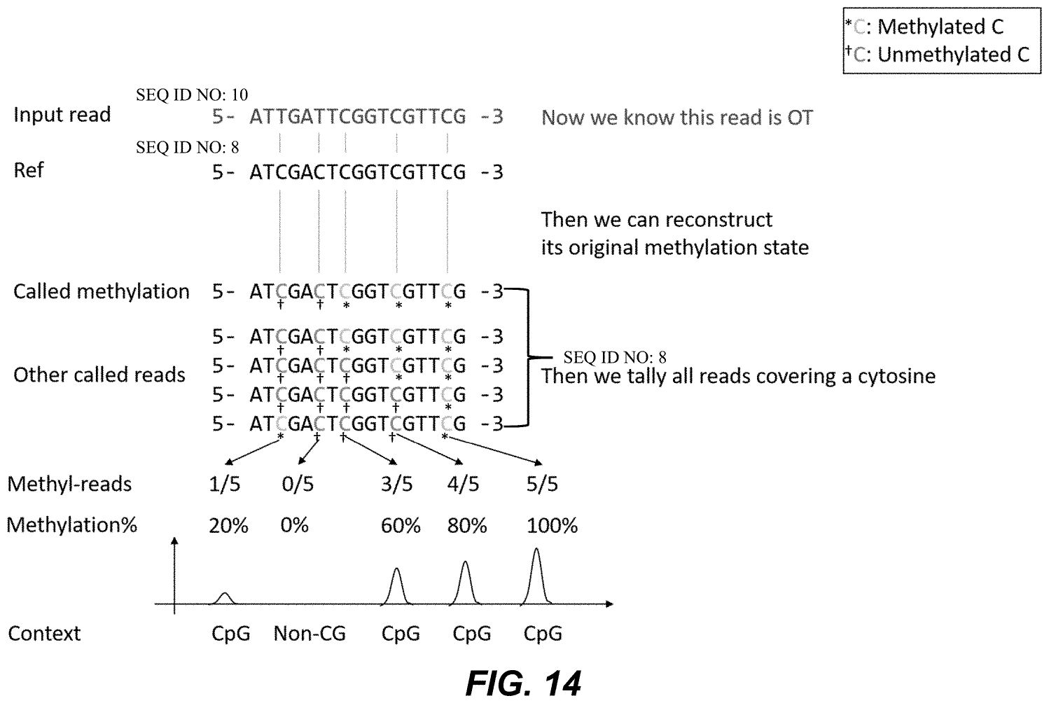

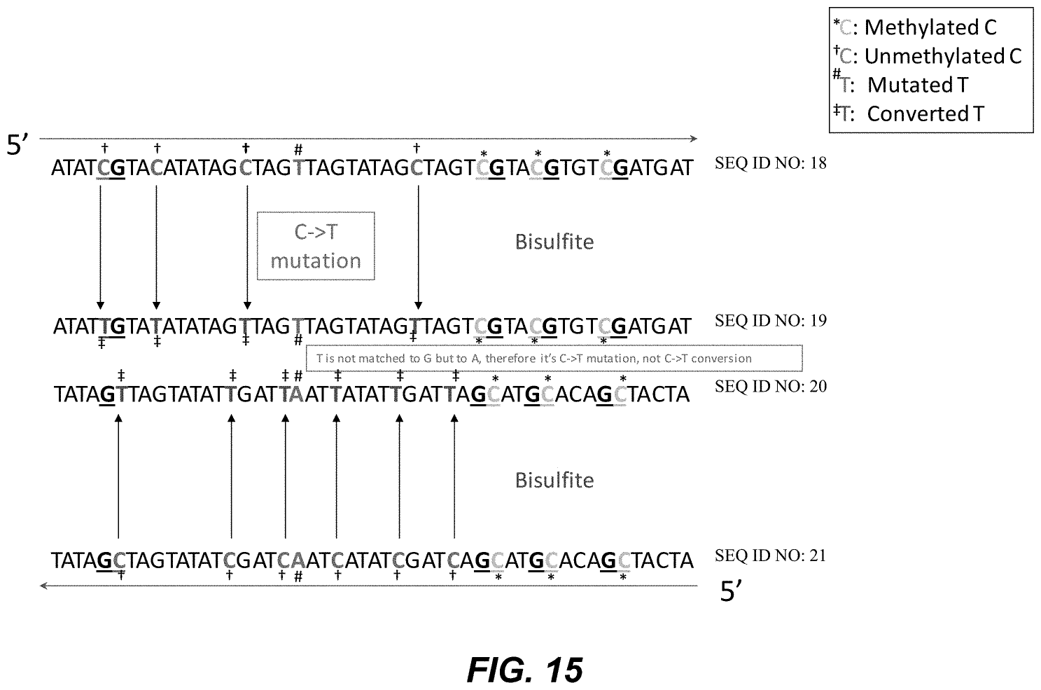

FIG. 12A-FIG. 12D depict non-limiting exemplary methylation assays (directional assay illustrated in FIG. 12A-FIG. 12C; non-directional assay illustrated in FIG. 12D) and sequence reads generated. Non-directional assays can comprise an extra PCR step (FIG. 12D). FIG. 13 illustrates methylation alignment and strand determination. FIG. 14 illustrates methylation calling following alignment. The methylation value can be called by tallying the total methylated C/(methylated+unmethylated C). CpG methylation can be symmetric and can be tallied from both directions. In a typical human genome, CpG methylation %: 75%, and Non-CG methylation %, <0.5%. Information from the original top strand and original bottom strand can be combined to distinguish C->T mutation vs C->T conversion when calling methylated sites. Multiple alignments can be performed to get information on both the forward and reverse strands, so that C->T conversion can be differentiated from C->T mutation (FIG. 15).

### Methylation Alignment and Calling Method

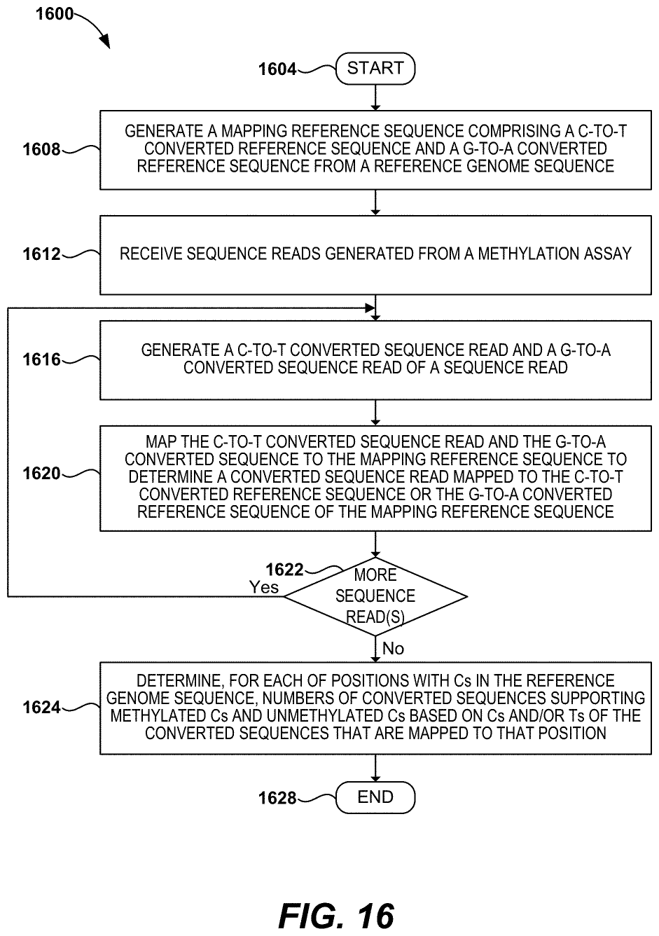

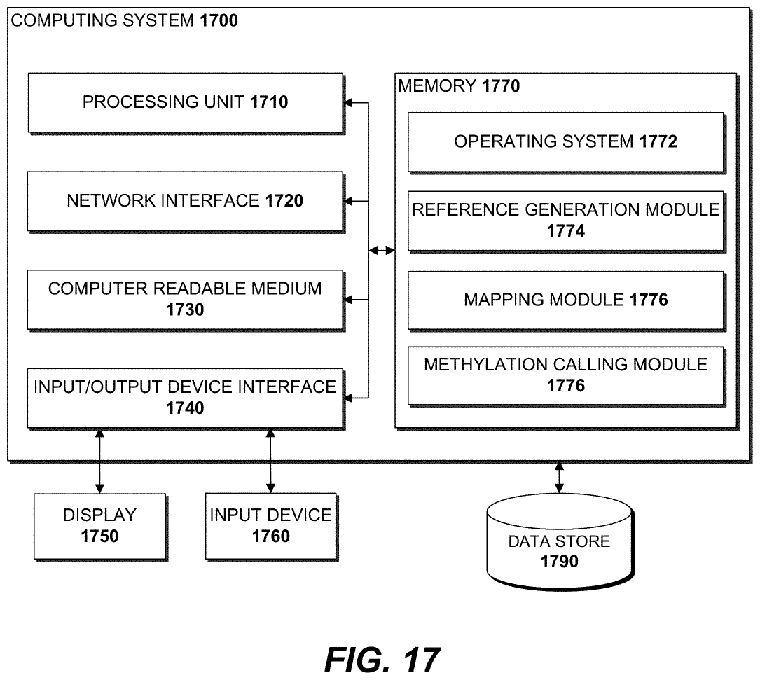

FIG. 16 is a flow diagram showing an exemplary method 1600 of a single-pass methylation calling method. A single-pass methylation calling method can be, for example, 2×, 2.5×, 3×, 3.5×, or 4× faster than a multi-pass methylation calling method. The method 1600 may be embodied in a set of executable program instructions stored on a computer-readable medium, such as one or more disk drives, of a computing system. For example, a system, machine, or device, such as the computing system 1700 shown in FIG. 17 and described in greater detail below, can include a processor (such as an FPGA or other programmable device) which executes a set of executable program instructions to implement the method 1600. When the method 1600 is initiated, the executable program instructions can be loaded into memory, such as RAM, and executed by one or more processors of the computing system 1700. Although the method 1600 is described with respect to the computing system 1700 shown in FIG. 17, the description is illustrative only and is not intended to be limiting. In some embodiments, the method 1600 or portions thereof may be performed serially or in parallel by multiple computing systems.

After the method 1600 begins at block 1604, the method 1600 proceeds to block 1608, where a system can generate a mapping reference sequence comprising a cytosine (C)-to-thymine (T) converted reference sequence and a guanine (G)-to-adenine (A) converted reference sequence from a reference genome sequence (or a reference sequence, which can be a reference genome sequence, or a portion thereof), such as hg19 or hg38. The reference genome sequence can be stored in the system's storage device. The system can receive the reference genome sequence used to generate the mapping reference sequence and stores the reference genome sequence in the system's storage device.

The mapping reference can comprise the C-to-T converted reference sequence and the G-to-A converted reference sequence with different reference identifiers. The C-to-T converted reference sequence can comprise all Cs in the reference genome sequence converted to Ts. The G-to-G converted reference sequence can comprise all Gs in the reference genome sequence converted to As. To generate the mapping reference sequence, the system can index the C-to-T converted reference sequence and the G-to-A converted reference sequence as two separate reference sequences together. The system can index the C-to-T converted reference sequence and the G-to-A converted reference sequence to generate a hashtable used for mapping. The hashtable can be about twice as large as a hashtable for one reference genome sequence.

The method 1600 proceeds from block 1608 to block 1612, where the system receives a plurality of sequence reads generated from a sample subjected to a methylation assay. The C-to-T converted sequence read can comprise all Cs in the sequence converted to Ts. The G-to-A converted sequence read can comprise all Gs in the sequence converted to As. The methylation assay can comprise bisulfide sequencing, whole genome-bisulfide sequencing (WGBS), Enzymatic Methyl-seq (EM-seq), or TET-assisted pyridine borane sequencing (TAPS). WGBS has been described in illumina.com/content/dam/illumina-marketing/documents/products/appnotes/appnote-methylseq-wgbs.pdf, the content of which is incorporated herein by reference in its entirety. EM-seq has been described in neb.com/-/media/nebus/files/manuals/manuale7120.pdf, the content of which is incorporated herein by reference in its entirety. TAPS has been described in Liu, et al. Bisulfite-free direct detection of 5-methylcytosine and 5-hydroxymethylcytosine at base resolution. Nat Biotechnol. 2019 April; 37(4):424-429. doi:10.1038/s41587-019-0041-2, the content of which is incorporated herein by reference in its entirety. The methylation assay can comprise a directional methylation protocol, a non-directional methylation protocol, or post-bisulfite adapter tagging (PBAT).

The plurality of sequence reads comprises sequence reads that are about 100 base pairs (bps) to about 1000 bps in length each, such as about 100 bps, 150 bps, 200 bps, 250 bps, 300 bps, 400 bps, 500 bps, 600 bps, 700 bps, 800 bps, 900 bps, or 1000 bps. The plurality of sequence reads can comprise paired-end sequence reads and/or single-end sequence reads. The plurality of sequence reads can be generated by whole genome sequencing (WGS), such as clinical WGS (cWGS). The sample can comprise cells, cell-free DNA, cell-free fetal DNA, amniotic fluid, a blood sample, a biopsy sample, or a combination thereof. The sample can be a sample of a subject. The sample can be obtained directly from a subject. The sample can be generated from another sample obtained from a subject. The other sample can be obtained directly from the subject.

The method 1600 proceeds from block 1612 to block 1616, where the system generates a C-to-T converted sequence read and a G-to-A converted sequence read from a sequence read of the plurality of sequence reads that has not been converted at block 1616 (and for which the corresponding C-to-T converted sequence read and G-to-A converted sequence read have not been mapped at block 1620). The converted sequence mapped to the C-to-T converted reference sequence or the G-to-A converted reference sequence of the mapping reference sequence can be (i) the C-to-T converted sequence, (ii) a reverse complement of the C-to-T converted sequence, (iii) the G-to-A converted sequence, or (iv) a reverse complement of the G-to-A converted sequence.

The method 1600 proceeds from block 1616 to block 1620, where the system map (or align) the C-to-T converted sequence read and the G-to-A converted sequence to the mapping reference sequence to determine a converted sequence read mapped to the C-to-T converted reference sequence or the G-to-A converted reference sequence of the mapping reference sequence. Run time does not change (or does not substantially change) with the mapping reference sequence being twice as large as the reference genome sequence.

The system can map the C-to-T converted sequence read and the G-to-A converted sequence to the mapping reference sequence to determine the best alignment amongst (i) the C-to-T converted sequence read and the C-to-T converted reference sequence, (ii) a reverse complement of the C-to-T converted sequence read and the C-to-T converted reference sequence, (iii) the G-to-A converted sequence read and the A-to-G converted reference sequence, and (iv) a reverse complement of the G-to-A converted sequence read and the G-to-A converted reference sequence. (i) The C-to-T converted sequence read, (ii) the reverse compliment of the C-to-T converted sequence read, (iii) the G-to-A converted sequence read, or (iv) the reverse compliment of the G-to-A converted sequence read resulting in the best alignment is the converted sequence read. The best alignment can have the highest alignment score. The best alignment can have no mismatch (or fewest mismatch(es)) in positions in the C-to-T converted reference genome or G-to-A converted reference genome corresponding to positions with Cs in the reference genome sequence to which (i) the C-to-T converted sequence, (ii) the reverse complement of the C-to-T converted sequence, (iii) the GC-to-A converted sequence, or (iv) the reverse complement of the G-to-A converted sequence is aligned.

The system can map the C-to-T converted sequence read and the G-to-A converted sequence read using a mapping algorithm that is or is similar to, for example, Bowtie, Bowtie2, or Burrows-Wheeler Aligner (BWA). The non-limiting examples of mapping algorithms include iSAAC, BarraCUDA, BFAST, BLASTN, BLAT, Bowtie, CASHX, Cloudburst, CUDA-EC, CUSHAW, CUSHAW2, CUSHAW2-GPU, drFAST, ELAND, ERNE, GNUMAP, GEM, GensearchNGS, GMAP and GSNAP, Geneious Assembler, LAST, MAQ, mrFAST and mrsFAST, MOM, MOSAIK, MPscan, Novoaligh & NovoalignCS, NextGENe, Omixon, PALMapper, Partek, PASS, PerM, PRIMEX, QPalma, RazerS, REAL, cREAL, RMAP, rNA, RT Investigator, Segemehl, SeqMap, Shrec, SHRiMP, SLIDER, SOAP, SOAP2, SOAP3 and SOAP3-dp, SOCS, SSAHA and SSAHA2, Stampy, SToRM, Subread and Subjunc, Taipan, UGENE, VelociMapper, XpressAlign, and ZOOM.

In some embodiments, to map the C-to-T converted sequence read and the G-to-A converted sequence to the mapping reference sequence, the system can determine the converted sequence read is a top strand (OT), a reverse complement of the top strand (CTOT), a bottom strand (OB), or a reverse complement of the bottom strand (CTOB) based on alignment types (See Table 2 for example alignment types).

In some embodiments, the system can, for each of one or more positions of the plurality of positions with Cs in the reference genome sequence to which a C or a T of the converted sequence read is mapped to, (i) increase a counter of C in that position if the converted sequence read comprises a C (which can indicate a methylated C) mapped to that position; and (ii) increasing a counter of T in that position if the converted sequence comprises a T which can indicate an unmethylated C) mapped to that position. Calling each of the plurality of positions with Cs in the reference genome sequence can comprise: calling each of the plurality of positions with Cs in the reference genome sequence is a methylated C or an unmethylated C based on (i) a value of the counter for T at that position and (ii) a value of the counter for C at that position. In some embodiments, steps (d1) and (d2) are performed by an aligner. Steps (d1), (d2), and (d3) can be performed by an aligner.

The method 1600 proceeds from block 1620 to decision block 1622. If there is at least one sequence read of the plurality of sequence reads that has not been converted at block 1616 (and for which the corresponding C-to-T converted sequence read and G-to-A converted sequence read have not been mapped at block 1620), the method 1600 proceeds from decision block 1622 to block 1616; otherwise the method 1600 proceeds to block 1624.

At block 1624, the system determines (or calls), for each of a plurality of positions with Cs in the reference genome sequence, (i) a number of the converted sequences that support methylated Cs and/or (ii) a number of the converted sequences that support unmethylated Cs at that position (of a strand of the reference genome sequence) based on (i) a number of Cs, if any, (which can indicate methylated Cs at that position in the sample) and/or (ii) a number of Ts, if any, (which can indicate unmethylated Cs at that position in the sample) of the converted sequences that are mapped to that position (of the strand of the reference genome sequence). The percentage of the converted sequences that support methylated Cs at that position can indicate the percentage of the DNA with methylated Cs at that position in the sample. The percentage of the converted sequences that support unmethylated Cs at that position can indicate the percentage of the DNA with unmethylated Cs at that position in the sample. The plurality of positions with Cs in the reference genome sequence can comprises at least 1,000 (or 5,000, 10,000, 50,000, 100,000, or more) positions, substantially all positions, or all positions with Cs in the reference genome sequence.

The system can determine (or call) (i) a percentage of the converted sequences that support methylated Cs (which can indicate the percentage of DNA with methylated Cs at that position in the sample) and/or (ii) a percentage of the converted sequences that support methylated Cs (which can indicate the percentage of DNA with unmethylated Cs at that position) using (i) a percentage of the converted sequences with Cs, if any, and/or (ii) a percentage of the converted sequences with Ts, if any, mapped to that position. The number of the converted sequences that support methylated Cs at that position (of the strand of the reference genome sequence) can be the number of Cs, if any, of the converted sequences that are mapped to that position (of the strand of the reference genome sequence). The number of the converted sequences that support unmethylated Cs at that position (of the strand of the reference genome sequence) can be the number of Ts, if any, of the converted sequences that are mapped to that position (of the strand of the reference genome sequence).

In some embodiments, the system can determine (or call), for each of one or more positions of the plurality of positions with Cs in the reference genome sequence, a number of the converted sequences that support C-to-T mutations based on a number of As of the converted sequences that are mapped to the complementary strand of the reference genome sequence at that position (and/or a number of Ts of the converted sequences that are mapped to the strand of the reference genome sequence at that position). The number of the converted sequences that support C-to-T mutations at that position can be a number of As of the converted sequences that are mapped to the complementary strand of the reference genome sequence at that position (or the minimum of the number of As of the converted sequences that are mapped to the complementary strand of the reference genome sequence at that position and the number of As of the converted sequences that are mapped to the complementary strand of the reference genome sequence at that position). The system can determine (or call), for each of one or more positions of the plurality of positions with Cs in the reference genome sequence, a percentage of the converted sequences that support C-to-T mutations at that position using a percentage of the converted sequences that are mapped to the complementary strand of the reference genome sequence with As at that position. The percentage of the converted sequences that support C-to-T mutations at that position can indicate the percentage of DNA with C-to-T mutations at that position in the sample

In some embodiments, the system creates a file or a report. The system can generate a user interface (UI) comprising a UI element. The file, report, or the UI element can represent or comprise the results of the method 1600. For example, the file, report, or the UI element can represent or comprise, for each of positions of the plurality of positions with Cs in the reference sequence, the number or percentage of Cs, if any, and/or the number or percentage of Ts, if any, of the converted sequences that are mapped to that position. As another example, the number or percentage of the converted sequences that support methylated Cs and/or the number or percentage of the converted sequences that support unmethylated Cs at that position. For example, the file, report, or the UI element can represent or comprise, for each of positions of the plurality of positions with Cs in the reference sequence, the percentage of DNA with methylated Cs at that position in the sample, the percentage of DNA with unmethylated Cs at that position in the sample, and/or the percentage of DNA with C-to-T mutations at that position in the sample. A UI element can be a window (e.g., a container window, browser window, text terminal, child window, or message window), a menu (e.g., a menu bar, context menu, or menu extra), an icon, or a tab. A UI element can be for input control (e.g., a checkbox, radio button, dropdown list, list box, button, toggle, text field, or date field). A UI element can be navigational (e.g., a breadcrumb, slider, search field, pagination, slider, tag, icon). A UI element can informational (e.g., a tooltip, icon, progress bar, notification, message box, or modal window). A UI element can be a container (e.g., an accordion).

The method 1600 ends at block 1628.

### Execution Environment

FIG. 17 depicts a general architecture of an example computing device 1700 configured to execute the processes and implement the features described herein. The general architecture of the computing device 1700 depicted in FIG. 17 includes an arrangement of computer hardware and software components. The computing device 1700 may include many more (or fewer) elements than those shown in FIG. 17. It is not necessary, however, that all of these generally conventional elements be shown in order to provide an enabling disclosure. As illustrated, the computing device 1700 includes a processing unit 1710, a network interface 1720, a computer readable medium drive 1730, an input/output device interface 1740, a display 1750, and an input device 1760, all of which may communicate with one another by way of a communication bus. The network interface 1720 may provide connectivity to one or more networks or computing systems. The processing unit 1710 may thus receive information and instructions from other computing systems or services via a network. The processing unit 1710 may also communicate to and from memory 1770 and further provide output information for an optional display 1750 via the input/output device interface 1740. The input/output device interface 1740 may also accept input from the optional input device 1760, such as a keyboard, mouse, digital pen, microphone, touch screen, gesture recognition system, voice recognition system, gamepad, accelerometer, gyroscope, or other input device.

The memory 1770 may contain computer program instructions (grouped as modules or components in some embodiments) that the processing unit 1710 executes in order to implement one or more embodiments. The memory 1770 generally includes RAM, ROM and/or other persistent, auxiliary or non-transitory computer-readable media. The memory 1770 may store an operating system 1772 that provides computer program instructions for use by the processing unit 1710 in the general administration and operation of the computing device 1700. The memory 1770 may further include computer program instructions and other information for implementing aspects of the present disclosure.

For example, in one embodiment, the memory 1770 includes a reference generation module 1774 which generates a mapping reference sequence comprising a C-to-T converted reference sequence and a G-to-A converted reference sequence from a reference genome sequence. The memory 1770 may additionally or alternatively include a mapping (or alignment) module 1776. The mapping module 1774 can (1) receive a sequence read, (2) generate a C-to-T converted sequence read and a G-to-A converted sequence read, and (3) map the C-to-T converted sequence read or the G-to-A converted sequence to the mapping reference sequence. The mapping module 1774 can repeat actions (1), (2), and (3) for sequence reads. The mapping module 1774 can repeat actions (1), (2), and (3) iteratively or in parallel for some sequence reads. The memory 1770 may additionally or alternatively include a methylation calling module 1778 for calling positions with Cs in the reference genome sequence is a methylated C or an unmethylated C in a sample from which the sequence read is generated. In addition, memory 1770 may include or communicate with the data store 1790 and/or one or more other data stores that the reference genome sequence, the sequence reads, and/or positions in the reference genome sequence with methylated Cs and/or unmethylated Cs in the sample called.

### Additional Considerations

In at least some of the previously described embodiments, one or more elements used in an embodiment can interchangeably be used in another embodiment unless such a replacement is not technically feasible. It will be appreciated by those skilled in the art that various other omissions, additions and modifications may be made to the methods and structures described above without departing from the scope of the claimed subject matter. All such modifications and changes are intended to fall within the scope of the subject matter, as defined by the appended claims.

One skilled in the art will appreciate that, for this and other processes and methods disclosed herein, the functions performed in the processes and methods can be implemented in differing order. Furthermore, the outlined steps and operations are only provided as examples, and some of the steps and operations can be optional, combined into fewer steps and operations, or expanded into additional steps and operations without detracting from the essence of the disclosed embodiments.

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity. As used in this specification and the appended claims, the singular forms “a,” “an,” and “the” include plural references unless the context clearly dictates otherwise. Accordingly, phrases such as “a device configured to” are intended to include one or more recited devices. Such one or more recited devices can also be collectively configured to carry out the stated recitations. For example, “a processor configured to carry out recitations A, B and C can include a first processor configured to carry out recitation A and working in conjunction with a second processor configured to carry out recitations B and C. Any reference to “or” herein is intended to encompass “and/or” unless otherwise stated.

It will be understood by those within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to embodiments containing only one such recitation, even when the same claim includes the introductory phrases “one or more” or “at least one” and indefinite articles such as “a” or “an” (e.g., “a” and/or “an” should be interpreted to mean “at least one” or “one or more”); the same holds true for the use of definite articles used to introduce claim recitations. In addition, even if a specific number of an introduced claim recitation is explicitly recited, those skilled in the art will recognize that such recitation should be interpreted to mean at least the recited number (e.g., the bare recitation of “two recitations,” without other modifiers, means at least two recitations, or two or more recitations). Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (e.g., “a system having at least one of A, B, or C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, such as in terms of providing a written description, all ranges disclosed herein also encompass any and all possible sub-ranges and combinations of sub-ranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like include the number recited and refer to ranges which can be subsequently broken down into sub-ranges as discussed above. Finally, as will be understood by one skilled in the art, a range includes each individual member. Thus, for example, a group having 1-3 articles refers to groups having 1, 2, or 3 articles. Similarly, a group having 1-5 articles refers to groups having 1, 2, 3, 4, or 5 articles, and so forth.

It will be appreciated that various embodiments of the present disclosure have been described herein for purposes of illustration, and that various modifications may be made without departing from the scope and spirit of the present disclosure. Accordingly, the various embodiments disclosed herein are not intended to be limiting, with the true scope and spirit being indicated by the following claims.

It is to be understood that not necessarily all objects or advantages may be achieved in accordance with any particular embodiment described herein. Thus, for example, those skilled in the art will recognize that certain embodiments may be configured to operate in a manner that achieves or optimizes one advantage or group of advantages as taught herein without necessarily achieving other objects or advantages as may be taught or suggested herein.

All of the processes described herein may be embodied in, and fully automated via, software code modules executed by a computing system that includes one or more computers or processors. The code modules may be stored in any type of non-transitory computer-readable medium or other computer storage device. Some or all the methods may be embodied in specialized computer hardware.

Many other variations than those described herein will be apparent from this disclosure. For example, depending on the embodiment, certain acts, events, or functions of any of the algorithms described herein can be performed in a different sequence, can be added, merged, or left out altogether (for example, not all described acts or events are necessary for the practice of the algorithms). Moreover, in certain embodiments, acts or events can be performed concurrently, for example through multi-threaded processing, interrupt processing, or multiple processors or processor cores or on other parallel architectures, rather than sequentially. In addition, different tasks or processes can be performed by different machines and/or computing systems that can function together.

The various illustrative logical blocks and modules described in connection with the embodiments disclosed herein can be implemented or performed by field programmable gate array (FPGA) discrete gate or transistor logic, discrete hardware components, or any combination thereof designed to perform the functions described herein. A processor can be a microprocessor, but in the alternative, the processor can be a controller, microcontroller, or state machine, combinations of the same, or the like. A processor can include electrical circuitry configured to process computer-executable instructions. In another embodiment, a processor includes an FPGA or other programmable device that performs logic operations without processing computer-executable instructions. A processor can also be implemented as a combination of computing devices, for example a combination of a DSP and a microprocessor, a plurality of microprocessors, one or more microprocessors in conjunction with a DSP core, or any other such configuration. Although described herein primarily with respect to digital technology, a processor may also include primarily analog components. For example, some or all of the signal processing algorithms described herein may be implemented in analog circuitry or mixed analog and digital circuitry. A computing environment can include any type of computer system, including, but not limited to, a computer system based on a microprocessor, a mainframe computer, a digital signal processor, a portable computing device, a device controller, or a computational engine within an appliance, to name a few.

Any process descriptions, elements or blocks in the flow diagrams described herein and/or depicted in the attached figures should be understood as potentially representing modules, segments, or portions of code which include one or more executable instructions for implementing specific logical functions or elements in the process. Alternate implementations are included within the scope of the embodiments described herein in which elements or functions may be deleted, executed out of order from that shown, or discussed, including substantially concurrently or in reverse order, depending on the functionality involved as would be understood by those skilled in the art.

It should be emphasized that many variations and modifications may be made to the above-described embodiments, the elements of which are to be understood as being among other acceptable examples. All such modifications and variations are intended to be included herein within the scope of this disclosure and protected by the following claims.

1. A method for methylation calling comprising:
under control of a hardware processor:
(a) receiving a reference genome sequence;
(b) generating a mapping reference sequence comprising a cytosine (C)-to-thymine (T) converted reference sequence and a guanine (G)-to-adenine (A) converted reference sequence from the reference genome sequence;
(c) receiving a plurality of sequence reads generated from a sample subjected to a methylation assay;
(d) for each of the plurality of sequence reads:
(d1) generating a C-to-T converted sequence read and a G-to-A converted sequence read; and
(d2) mapping the C-to-T converted sequence read and the G-to-A converted sequence read to the mapping reference sequence to determine a converted sequence read mapped to the C-to-T converted reference sequence or the G-to-A converted reference sequence of the mapping reference sequence; and
(e) determining, for each of a plurality of positions with Cs in the reference genome sequence, a number of the converted sequences that support methylated Cs and/or a number of the converted sequences that support unmethylated Cs at that position based on a number of Cs, if any, and/or a number of Ts, if any, of the converted sequences that are mapped to that position.

2. (canceled)

3. (canceled)

4. The method of claim 1, wherein generating the mapping reference sequence comprises: indexing the C-to-T converted reference sequence and the G-to-A converted reference sequence as two separate reference sequences together, optionally wherein the mapping reference comprises the C-to-T converted reference sequence and the G-to-A converted reference sequence with different reference identifiers.

5. The method of claim 1, wherein the C-to-T converted reference sequence comprises all Cs in the reference genome sequence converted to Ts, and wherein the G-to-A converted reference sequence comprises all Gs in the reference genome sequence converted to As; and/or wherein the C-to-T converted sequence read comprises all Cs in the sequence converted to Ts, and wherein the G-to-A converted sequence read comprises all Gs in the sequence converted to As.

6. (canceled)

7. The method of claim 1, wherein the converted sequence is the C-to-T converted sequence, a reverse complement of the C-to-T converted sequence, the G-to-A converted sequence, or a reverse complement of the G-to-A converted sequence.

8. The method of claim 1, wherein mapping the C-to-T converted sequence read and the G-to-A converted sequence read to the mapping reference sequence comprises: mapping the C-to-T converted sequence read and the G-to-A converted sequence read to the mapping reference sequence to determine the best alignment amongst (i) the C-to-T converted sequence read and the C-to-T converted reference sequence, (ii) a reverse complement of the C-to-T converted sequence read and the C-to-T converted reference sequence, (iii) the G-to-A converted sequence read and the G-to-A converted reference sequence, and (iv) a reverse complement of the G-to-A converted sequence read and the G-to-A converted reference sequence, and wherein the C-to-T converted sequence read, the reverse compliment of the C-to-T converted sequence read, the G-to-A converted sequence read, or the reverse compliment of the G-to-A converted sequence read resulting in the best alignment is the converted sequence read.

9. The method of claim 1, wherein mapping the C-to-T converted sequence read and the G-to-A converted sequence read to the mapping reference sequence comprises determining the converted sequence read is a top strand, a reverse complement of the top strand, a bottom strand, or a reverse complement of the bottom strand based on alignment types.

10. The method of claim 1, wherein the number of the converted sequences that support methylated Cs at that position is the number of Cs, if any, of the converted sequences that are mapped to that position, and wherein the number of the converted sequences that support unmethylated Cs at that position is the number of Ts, if any, of the converted sequences that are mapped to that position.

11. The method of claim 1,
wherein determining, for each of the plurality of positions with Cs in the reference genome sequence, a number of the converted sequences that support methylated Cs and/or a number of the converted sequences that support unmethylated Cs at that position comprises: determining (i) a percentage of the converted sequences that support methylated Cs and/or (ii) a percentage of the converted sequences that support methylated Cs using (i) a percentage of the converted sequences with Cs, if any, and/or (ii) a percentage of the converted sequences with Ts, if any, mapped to that position, and/or
wherein the percentage of the converted sequences that support methylated Cs at that position indicate the percentage of the DNA with methylated Cs at that position in the sample, and wherein the percentage of the converted sequences that support unmethylated Cs at that position indicate the percentage of the DNA with unmethylated Cs at that position in the sample.

12. The method of claim 1, wherein determining, for each of the plurality of positions with Cs in the reference genome sequence, the number of the converted sequences that support methylated Cs and/or the number of the converted sequences that support unmethylated Cs at that position comprises: determining, for each of one or more positions of the plurality of positions with Cs in the reference genome sequence, a number of the converted sequences that support C-to-T mutations based on a number of As of the converted sequences that are mapped to the complementary strand of the reference genome sequence at that position.

13. The method of claim 12, wherein the number of the converted sequences that support C-to-T mutations at that position is a number of As of the converted sequences that are mapped to the complementary strand of the reference genome sequence at that position, and/or wherein determining, for each of one or more positions of the plurality of positions with Cs in the reference genome sequence, the number of the converted sequences that support C-to-T mutations comprises: determining, for each of one or more positions of the plurality of positions with Cs in the reference genome sequence, a percentage of the converted sequences that support C-to-T mutations at that position using a percentage of the converted sequences that are mapped to the complementary strand of the reference genome sequence with As at that position, optionally wherein the percentage of the converted sequences that support C-to-T mutations at that position indicate the percentage of DNA with C-to-T mutations at that position in the sample.

14. The method of claim 1, wherein the plurality of positions with Cs in the reference genome sequence comprises at least 10,000 positions, substantially all positions, or all positions with Cs in the reference genome sequence.

15. The method of claim 1,
further comprising: (d3) for each of one or more positions of the plurality of positions with Cs in the reference genome sequence to which a C or a T of the converted sequence read is mapped to, (i) increasing a counter of C in that position if the converted sequence read comprises a C mapped to that position; and (ii) increasing a counter of T in that position if the converted sequence comprises a T mapped to that position,
wherein determining, for each of the plurality of positions with Cs in the reference genome sequence, the number of the converted sequences that support methylated Cs and/or the number of the converted sequences that support unmethylated Cs at that position comprises: determining, for each of the plurality of positions with Cs in the reference genome sequence, the number of the converted sequences that support methylated Cs and/or the number of the converted sequences that support unmethylated Cs at that position based on (i) a value of the counter for C at that position and (ii) a value of the counter for T at that position.

16. The method of claim 1, wherein steps (d1) and (d2) are performed by an aligner, or wherein steps (d1), (d2), and (d3) are performed by an aligner.

17. The method of claim 1, wherein the method is at least 2× faster than a multi-pass methylation calling method.

18. The method of claim 1, wherein the methylation assay comprises bisulfate sequencing, whole genome-bisulfite sequencing (WGBS), Enzymatic Methyl-seq (EM-seq), or TET-assisted pyridine borane sequencing (TAPS), and/or wherein the methylation assay comprises a directional methylation protocol, a non-directional methylation protocol, or post-bisulfate adapter tagging (PBAT); and wherein the plurality of sequence reads is generated by whole genome sequencing (WGS), optionally wherein the WGS is clinical WGS (cWGS).

19. The method of claim 1, further comprising: creating a file or a report and/or generating a user interface (UI) comprising a UI element representing or comprising, for each of positions of the plurality of positions with Cs in the reference genome sequence, the number or percentage of the converted sequences that support methylated Cs and/or the number or percentage of the converted sequences that support unmethylated Cs at that position.

20. The method of claim 1, wherein the plurality of sequence reads comprises sequence reads that are about 100 base pairs to about 1000 base pairs in length each, and/or wherein the plurality of sequence reads comprises paired-end sequence reads and/or single-end sequence reads.

21. (canceled)

22. (canceled)

23. The method of claim 1, wherein the sample comprises cells, cell-free DNA, cell-free fetal DNA, amniotic fluid, a blood sample, a biopsy sample, or a combination thereof.

24. The method of claim 1, wherein the sample is a sample of a subject, optionally wherein the sample is obtained directly from a subject.

25. (canceled)

26. (canceled)

27. A system for methylation calling comprising:
non-transitory memory configured to store executable instructions, and a mapping reference sequence comprising a cytosine (C)-to-thymine (T) converted reference sequence and a guanine (G)-to-adenine (A) converted reference sequence generated from a reference sequence; and
a hardware processor in communication with the non-transitory memory, the hardware processor programmed by the executable instructions to perform:
(a) receiving a plurality of sequence reads generated from a sample subjected to a methylation assay;
(b) for each of the plurality of sequence reads:
(b1) generating a C-to-T converted sequence read and a G-to-A converted sequence read; and
(b2) mapping the C-to-T converted sequence read and the G-to-A converted sequence read to the mapping reference sequence to determine a converted sequence read mapped to the C-to-T converted reference sequence or the G-to-A converted reference sequence of the mapping reference sequence; and
(c) determining, for each of a plurality of positions with Cs in the reference genome sequence, a number of the converted sequences that support methylated Cs and/or a number of the converted sequences that support unmethylated Cs at that position based on a number of Cs, if any, and/or a number of Ts, if any, of the converted sequences that are mapped to that position.

28.-53. (canceled)


SEQUENCE LISTING (21 sequences)

SEQ ID NO: 1 - DNA, 18 bp, synthetic construct
  misc_feature 1..18: note = genomic fragment
  source 1..18: mol_type = other DNA
ccggcatgtttaaacgct

SEQ ID NO: 2 - DNA, 18 bp, synthetic construct
  misc_feature 1..18: note = genomic fragment-bisulfite treated
  source 1..18: mol_type = other DNA
ttggcatgtttaaacgtt

SEQ ID NO: 3 - DNA, 18 bp, synthetic construct
  misc_feature 1..18: note = c to t converted
  source 1..18: mol_type = other DNA
ttggtatgtttaaatgtt

SEQ ID NO: 4 - DNA, 18 bp, synthetic construct
  misc_feature 1..18: note = g to a converted
  source 1..18: mol_type = other DNA
ttaacatatttaaacatt

SEQ ID NO: 5 - DNA, 18 bp, synthetic construct
  misc_feature 1..18: note = forward g to a
  source 1..18: mol_type = other DNA
ccaacatatttaaacact

SEQ ID NO: 6 - DNA, 18 bp, synthetic construct
  misc_feature 1..18: note = converted genome c to t
  source 1..18: mol_type = other DNA
aacatttaaacataccaa

SEQ ID NO: 7 - DNA, 18 bp, synthetic construct
  misc_feature 1..18: note = converted genome g to a
  source 1..18: mol_type = other DNA
agtgtttaaatatgttgg

SEQ ID NO: 8 - DNA, 17 bp, synthetic construct
  misc_feature 1..17: note = top strand
  source 1..17: mol_type = other DNA
atcgactcggtcgttcg

SEQ ID NO: 9 - DNA, 17 bp, synthetic construct
  misc_feature 1..17: note = bottom strand
  source 1..17: mol_type = other DNA
cgaacgaccgagtcgat

SEQ ID NO: 10 - DNA, 17 bp, synthetic construct
  misc_feature 1..17: note = original top
  source 1..17: mol_type = other DNA
attgattcggtcgttcg

SEQ ID NO: 11 - DNA, 17 bp, synthetic construct
  misc_feature 1..17: note = original bottom
  source 1..17: mol_type = other DNA
cgaacgatcgagttgat

SEQ ID NO: 12 - DNA, 17 bp, synthetic construct
  misc_feature 1..17: note = Complement of original bottom
  source 1..17: mol_type = other DNA
atcaactcgatcgttcg

SEQ ID NO: 13 - DNA, 17 bp, synthetic construct
  misc_feature 1..17: note = Complement of original top
  source 1..17: mol_type = other DNA
cgaacgaccgaatcaat

SEQ ID NO: 14 - DNA, 17 bp, synthetic construct
  misc_feature 1..17: note = Read c to t forward
  source 1..17: mol_type = other DNA
attgatttggttgtttg

SEQ ID NO: 15 - DNA, 17 bp, synthetic construct
  misc_feature 1..17: note = Read g to a Forward
  source 1..17: mol_type = other DNA
attaattcaatcattca

SEQ ID NO: 16 - DNA, 17 bp, synthetic construct
  misc_feature 1..17: note = Read c to t reverse
  source 1..17: mol_type = other DNA
attaatttgattgtttg

SEQ ID NO: 17 - DNA, 17 bp, synthetic construct
  misc_feature 1..17: note = Read g to a reverse
  source 1..17: mol_type = other DNA
atcaactcaatcattca

SEQ ID NO: 18 - DNA, 49 bp, synthetic construct
  misc_feature 1..49: note = Forward
  source 1..49: mol_type = other DNA
atatcgtacatatagctagttagtatagctagtcgtacgtgtcgatgat

SEQ ID NO: 19 - DNA, 49 bp, synthetic construct
  misc_feature 1..49: note = Bisulfite forward
  source 1..49: mol_type = other DNA
atattgtatatatagttagttagtatagttagtcgtacgtgtcgatgat

SEQ ID NO: 20 - DNA, 49 bp, synthetic construct
  misc_feature 1..49: note = Bisulfite reverse
  source 1..49: mol_type = other DNA
atcatcgacacgtacgattagttatattaattagttatatgattgatat

SEQ ID NO: 21 - DNA, 49 bp, synthetic construct
  misc_feature 1..49: note = Reverse
  source 1..49: mol_type = other DNA
atcatcgacacgtacgactagctatactaactagctatatgatcgatat